(19) United States
(12) Patent Application Publication
(10) Pub. No.: US 2017/0246222 A1
(43) Pub. Date: Aug. 31, 2017

(54) Bacillus Strains with Fast Germination and Antimicrobial Activity against Clostridium

(71) Applicant: Novozymes A/S, Bagsvaerd (DK)

(72) Inventors: Derrick Lewis, Durham, NC (US); Preben Nielsen, Horsholm (DK); Dorthe Hoej Sandvang, Slangerup (DK); Adam Nelson, Salem, VA (US)

(73) Assignee: Novozymes A/S, Bagsvaerd (DK)

(21) Appl. No.: 15/514,941

(22) PCT Filed: Oct. 8, 2015
(86) PCT No.: PCT/US15/54715

§ 371 Date: Mar. 28, 2017

Related U.S. Application Data
(60) Provisional application No. 62/061,300, filed on Oct. 8, 2014.

(51) Int. Cl. A61K 35/74; A23K 50/75; A23K 10/18; A23K 50/30; C12R 1/125; C12R 1/07

(52) U.S. Cl. CPC A61K 35/74; C12R 1/125; C12R 1/07; A23K 10/18; A23K 50/30; A23K 50/75; A61K 2035/11

(57) ABSTRACT

The present invention relates to compositions such as an animal feed comprising one or more bacteria with fast germination and with anti-Clostridium perfringens effect.

### REFERENCE TO A DEPOSIT OF BIOLOGICAL MATERIAL

This application contains a reference to a deposit of biological material, which deposit is incorporated herein by reference.

### BACKGROUND OF THE INVENTION

Field of the Invention

The present invention relates to compositions such as an animal feed comprising one or more bacteria with fast germination and/or with antimicrobial activity against Clostridium perfringens.

Background of the Invention

Clostridium perfringens (C. perfringens) is a Gram-positive, rod-shaped, anaerobic, spore-forming bacterium of the genus Clostridium. C. perfringens is widely present in nature and can be found as a component of decaying vegetation, marine sediment, the intestinal tract of humans and other vertebrates, insects, and soil.

Infections due to C. perfringens show evidence of tissue necrosis, bacteremia, emphysematous cholecystitis, and gas gangrene, which is also known as clostridial myonecrosis. C. perfringens can also result in polymicrobial anaerobic infections.

The incidence of Clostridium perfringens-associated necrotic enteritis in poultry has increased in countries that stopped using antibiotic growth promoters. Necrotic enteritis is an enterotoxemic disease that results in significant economic losses in the poultry industry.

There is a need for development of tools and strategies for prevention and control of C. perfringens in mono-gastric animals such as poultry. Vaccination against the pathogen and the use of probiotic and prebiotic products has been suggested.

It is thus an object of the invention to provide solutions which prevents and/or controls C. perfringens in poultry and/or swine by use of an animal feed comprising one or more bacteria with improved intestinal germination and with anti-Clostridium perfringens effect.

### DESCRIPTION OF THE RELATED ART

WO 2010/033714 describes a method for enhancing the health of an animal comprising administering to the animal a composition comprising Bacillus subtilis QST713.

U.S. Pat. No. 4,919,936 describes a method for increasing the weight gain in animals comprising feeding an animal a probiotic comprising Bacillus subtilis C-3102.

Knap et al. (Knap et al., 2010, Bacillus licheniformis Prevents Necrotic Enteritis in Broiler Chickens, Avian Diseases 54(2): 931-935) describes that Bacillus licheniformis has an effect on necrotic enteritis in broiler chickens.

### SUMMARY OF THE INVENTION

It has been surprisingly found that the addition of direct fed microbes (DFM) from Bacillus species to animal feed can be used to treat and/or prevent and/or control C. perfringens infections and/or necrotic enteritis in mono-gastric animal such as pigs and/or poultry.

In one embodiment an animal feed composition comprising spores of a Bacillus strain wherein:

i. the Bacillus strain is a Bacillus subtilis strain or a Bacillus amyloliquefaciens strain;

ii. at least 60% of the Bacillus spores survive gastric stability (e.g. as determined in Example 1);

iii. at least 55% of the Bacillus spores germinate in less than 4 hours (e.g. as determined in Example 1),

iv. the Bacillus spores have antimicrobial activity e.g. against Clostridium perfringens (e.g. as determined in Example 1) and

v. the Bacillus strain has enzyme activity under aerobic conditions against one or more of the substrates selected from the group consisting of Amylose, Arabinan, Arabinoxylan, Casein and Xylan (e.g. as determined in Example 1). The Bacillus strains can furthermore form biofilm in vitro as determined in Example 2 and/or have in vitro mucin adhesion properties as determined in Example 2.

The invention further relates to a method for treating and/or preventing and/or controlling C. perfringens infections and/or necrotic enteritis in a mono-gastric animal such as swine or poultry by use of an animal feed, wherein the animal feed comprises one or more Bacillus strains selected from the group consisting of the strain having the deposit accession number NRRL B-50136; NRRL B-50147; NRRL B-50141; PTA-7543; NRRL B-50888; PTA-7549; and NRRL B-50349 (or a strain having all of the identifying characteristics of any of the strains or a mutant of any of the strains), or any combination thereof, wherein the Bacillus strain has antimicrobial activity against Clostridium perfringens.

In an embodiment, the invention relates to an animal feed that is fed to a mono-gastric animal such as poultry or swine; comprising one or more Bacillus strains selected from the group consisting of the strain having the deposit accession number NRRL B-50136; NRRL B-50147; NRRL B-50141; PTA-7543; NRRL B-50888; PTA-7549; and NRRL B-50349 (or a strain having all of the identifying characteristics of any of the strains or a mutant of any of the strains), or any combination thereof, wherein the Bacillus strain has antimicrobial activity against Clostridium perfringens.

In a preferred embodiment the invention relates to an animal feed composition such as a swine or poultry animal feed composition comprising a Bacillus strain wherein:

i. at least 60% of the Bacillus spores survive gastric stability; and/or

ii. at least 55% of the Bacillus spores germinate in less than 4 hours, and

iii. the Bacillus strain is selected from the group consisting of:
the strain having the deposit accession number NRRL B-50136;
the strain having the deposit accession number NRRL B-50147;
the strain having the deposit accession number NRRL B-50141;
the strain having the deposit accession number PTA-7543;
the strain having the deposit accession number NRRL B-50888;
the strain having the deposit accession number PTA-7549; and
the strain having the deposit accession number NRRL B-50349 (or a strain having all of the identifying characteristics of any of the strains or a mutant of any of the strains), or any combination thereof and

iv. the Bacillus spores have antimicrobial activity against Clostridium perfringens.

In a further aspect, the invention relates to a method for treating and/or preventing and/or controlling C. perfringens infections and/or necrotic enteritis in an animal such as pigs or poultry, comprising the steps of:

(a) feeding an animal (such as a pig or poultry) a feed (such as a pig feed or a poultry feed); and

(b) administering to an animal such as pigs or poultry one or more Bacillus strains or spores of the strains selected from the group consisting of the strain having the deposit accession number ATCC 700385, NRRL B-50136, NRRL B-50147, NRRL B-50622, NRRL B-50623, NRRL B-50605, NRRL B-50621, NRRL B-50015, NRRL B-50016, NRRL B-50885, NRRL B-50886, NRRL B-50141, NRRL B-50151, NRRL B-50606, PTA-7543, PTA-7547, NRRL B-50888, PTA-7549 and NRRL B-50349 (or a strain having all of the identifying characteristics of any of the strains or a mutant of any of the strains), or any combination thereof, wherein the Bacillus strain has antimicrobial activity against Clostridium perfringens, and wherein step (a) occurs before, after, or simultaneously with step (b). In an embodiment, the method relates to an animal feed that is fed to a mono-gastric animal such as pigs and/or poultry. The poultry can e.g. be chickens and/or broilers and/or layers.

In an embodiment, the animal feed composition comprises one of more additional bacteria; is fed to a mono-gastric animal such as pigs and/or poultry; and further comprises concentrate and/or one or more enzymes and/or one or more additional microbes and/or one or more vitamins and/or one or more minerals and/or one or more amino acids and/or one or more other feed ingredients.

### FIGURE LEGENDS

FIG. 1: In vitro Screening of Anti-Clostridium perfringens Activity in NRRL B50349 and ATCC PTA-6737. Three top plates are two Radial diffusion plates with triplicate test of NRRL B50349, third top plate is cross streaking with NRRL B50349. The three plates below are ATCC PTA-6737 tested the same way.

FIG. 2: Biofilm formation is shown as the white pellicle at the liquid:air interface in strains ATCC PTA-7549 (left) and NRRL B-50349 (right).

### DEFINITIONS

In general, the terms and phrases used herein have their art-recognized meaning, which can be found by reference to standard texts, journal references, and context known to those skilled in the art. The following definitions are provided to clarify their specific use in context of the disclosure.

As used herein, the singular forms “a”, “an” and “the” are intended to include the plural forms as well, unless the context clearly indicates otherwise.

Animal feed: The term “animal feed” refers to any compound, preparation, or mixture suitable for, or intended for intake by an animal. Animal feed for a mono-gastric animal comprises concentrates as well as vitamins, minerals, enzymes, amino acids and/or other feed ingredients (such as in a premix). The animal feed may further comprise forage.

Antimicrobial activity against Clostridium perfringens: The term “Antimicrobial activity against Clostridium perfringens” means that the growth of Clostridium perfringens is inhibited and/or that some or all of the Clostridium perfringens are killed. This can be determined by the assay described in Example 1.

Blend: the term “blend” means more than one of the bacterial strains described herein.

Composition: The term “composition” refers to a composition comprising a carrier and at least one bacterial strain as described herein. The compositions described herein may be mixed with an animal feed(s) and referred to as a “mash feed.”

Concentrates: The term “concentrates” means feed with high protein and energy concentrations, such as fish meal, molasses, oligosaccharides, sorghum, seeds and grains (either whole or prepared by crushing, milling, etc from e.g. corn, oats, rye, barley, wheat), oilseed press cake (e.g. from cottonseed, safflower, sunflower, soybean, rapeseed/canola, peanut or groundnut), palm kernel cake, yeast derived material and distillers grains (such as wet distillers grains (WDS) and dried distillers grains with solubles (DDGS)).

Control C. perfringens infections and/or necrotic enteritis: The term “control C. perfringens infections and/or necrotic enteritis” means a method and/or composition that partly or completely inhibits C. perfringens infections and/or necrotic enteritis in an animal. Accordingly, the term “control C. perfringens infections and/or necrotic enteritis” means the C. perfringens infections and/or the necrotic enteritis is reduced or completely eliminated.

Direct Fed Microbial: The term “direct fed microbial” means live micro-organisms including spores which, when administered in adequate amounts, confer a benefit, such as improved digestion or health, on the host.

Effective amount/concentration/dosage: The terms “effective amount”, “effective concentration”, or “effective dosage” are defined as the amount, concentration, or dosage of the bacterial strain(s) sufficient to improve the digestion or yield of an animal. The actual effective dosage in absolute numbers depends on factors including: the state of health of the animal in question, other ingredients present. The “effective amount”, “effective concentration”, or “effective dosage” of the bacterial strains may be determined by routine assays known to those skilled in the art.

Forage: The term “forage” as defined herein also includes roughage. Forage is fresh plant material such as hay and silage from forage plants, grass and other forage plants, seaweed, sprouted grains and legumes, or any combination thereof. Examples of forage plants are Alfalfa (lucerne), birdsfoot trefoil, brassica (e.g. kale, rapeseed (canola), rutabaga (swede), turnip), clover (e.g. alsike clover, red clover, subterranean clover, white clover), grass (e.g. Bermuda grass, brome, false oat grass, fescue, heath grass, meadow grasses, orchard grass, ryegrass, Timothy-grass), corn (maize), millet, barley, oats, rye, sorghum, soybeans and wheat and vegetables such as beets. Forage further includes crop residues from grain production (such as corn stover; straw from wheat, barley, oat, rye and other grains); residues from vegetables like beet tops; residues from oilseed production like stems and leaves form soy beans, rapeseed and other legumes; and fractions from the refining of grains for animal or human consumption or from fuel production or other industries.

Gastric survival: The terms “gastric survival” and “survive gastric stability” are defined as the % of surviving spores measured by the assay and conditions described in Example 1.

Germinate in less than 4 hours: The term “germination in less than 4 hours” means the % of spores that germinate in less than 4 hours measured by the assay and conditions described in Example 1.

Isolated: The term “isolated” means that the one or more bacterial strains described herein are in a form or environment which does not occur in nature, that is, the one or more bacterial strains are at least partially removed from one or more or all of the naturally occurring constituents with which it is associated in nature.

Pellet: The terms “pellet” and/or “pelleting” refer to solid rounded, spherical and/or cylindrical tablets or pellets and the processes for forming such solid shapes, particularly feed pellets and solid extruded animal feed. As used herein, the terms “extrusion” or “extruding” are terms well known in the art and refer to a process of forcing a composition, as described herein, through an orifice under pressure.

Poultry: The term “poultry” means domesticated birds kept by humans for the eggs they produce and/or their meat and/or their feathers. Poultry includes broilers and layers. Poultry include members of the superorder Galloanserae (fowl), especially the order Galliformes (which includes chickens, Guineafowls, quails and turkeys) and the family Anatidae, in order Anseriformes, commonly known as “waterfowl” and including domestic ducks and domestic geese. Poultry also includes other birds that are killed for their meat, such as the young of pigeons. Examples of poultry include chickens (including layers, broilers and chicks), ducks, geese, pigeons, turkeys and quail.

Prevent C. perfringens infections and/or necrotic enteritis: The term “prevent C. perfringens infections and/or necrotic enteritis” means a method and/or composition that prevents development of a C. perfringens infection and/or necrotic enteritis in an animal.

Roughage: The term “roughage” means dry plant material with high levels of fiber, such as fiber, bran, husks from seeds and grains and crop residues (such as stover, copra, straw, chaff, sugar beet waste).

Silage: The term “silage” means fermented, high-moisture stored fodder which can be fed to ruminants (cud-chewing animals such as cattle and sheep) or used as a biofuel feedstock for anaerobic digesters. It is fermented and stored in a process called ensilage, ensiling or silaging, and is usually made from grass or cereal crops (e.g. maize, sorghum, oats, rye, timothy etc forage grass plants),) or legume crops like clovers/trefoils, alfalfa, vetches, using the entire green plant (not just the grain). Silage can be made from many field crops, and special terms may be used depending on type (oatlage for oats, haylage for alfalfa). Silage is made either by placing cut green vegetation in a silo, by piling it in a large heap covered with plastic sheet, or by wrapping large bales in plastic film.

Spore: The terms “spore” and “endospore” are interchangeable and have their normal meaning which is well known and understood by those of skill in the art. As used herein, the term spore refers to a microorganism in its dormant, protected state.

Stable: The term “stable” is a term that is known in the art, and in a preferred aspect, stable is intended to mean the ability of the microorganism to remain in a spore form until it is administered to an animal to improve the health of the animal.

Sub-optimal animal feed: The terms “sub-optimal animal feed” and “animal feed is sub-optimal” means an animal feed that contains a high level of un-digestible organic matter, such as the animal feed comprises at least 30%, preferably at least 40%, more preferably at least 50%, even more preferably at least 60% and most preferably at least 70% un-digestible organic matter. Organic Matter (OM) digestibility for ruminants of different types of animal feed can be obtained by looking at reference texts such as e.g. www.feedipedia.org.

Swine: The term “swine” or “pigs” means domesticated pigs kept by humans for food, such as their meat. Swine includes members of the genus Sus, such as Sus scrofa domesticus or Sus domesticus and include piglets, growing pigs, and sows. Swine and pigs are used interchangeably herein.

Treat C. perfringens infections and/or necrotic enteritis: the treatment can be ameliorating and/or curative and/or prophylactic.

Vegetable protein: The term “vegetable protein” refers to any compound, preparation or mixture that includes at least one protein derived from or originating from a vegetable, including modified proteins and protein-derivatives.

### DETAILED DESCRIPTION OF THE INVENTION

### Compositions

In one aspect the invention relates to an animal feed composition comprising spores of a Bacillus strain wherein:

i. the Bacillus strain is a Bacillus subtilis strain or a Bacillus amyloliquefaciens strain;

ii. at least 60% (such as at least 65%, at least 70%, at least 80% or at least 90%) of the Bacillus spores survive gastric stability (e.g. determined as described in Example 1);

iii. at least 55% (such as at least 60%, at least 70%, at least 80% or at least 90%) of the Bacillus spores germinate in less than 4 hours (e.g. determined as described in Example 1),

iv. the Bacillus spores have antimicrobial activity against Clostridium perfringens and

v. the Bacillus strain has enzyme activity under aerobic conditions against one or more of the substrates selected from the group consisting of amylose, arabinan, arabinoxylan, casein and xylan (e.g. determined as described in Example 1).

In one embodiment the Bacillus strain has enzyme activity under aerobic conditions against one or more substrates selected from the group consisting of amylose, arabinan, arabinoxylan, casein, cellulose and xylan e.g. determined as described in Example 1.

In one embodiment the Bacillus strain is a Bacillus amyloliquefaciens strain and has enzyme activity under aerobic conditions against one or more substrates selected from the group consisting of amylose, arabinan, arabinoxylan, casein, cellulose and xylan (e.g. determined as described in Example 1).

In one embodiment the Bacillus strain is a Bacillus subtilis strain and has enzyme activity under aerobic conditions against one or more substrates selected from the group consisting of amylose, arabinan, arabinoxylan, casein and xylan e.g. determined as described in Example 1.

In one embodiment the Bacillus strain has enzyme activity under anaerobic conditions against one or more substrates selected from the group consisting of amylose, arabinan, arabinoxylan, casein, cellulose and xylan e.g. determined as described in Example 1.

In one embodiment the Bacillus strain is a Bacillus amyloliquefaciens strain and has enzyme activity under anaerobic conditions against one or more substrates selected from the group consisting of amylose, arabinan, arabinoxylan, casein, cellulose and xylan e.g. determined as described in Example 1.

In one embodiment the Bacillus strain is a Bacillus subtilis strain and has enzyme activity under anaerobic conditions against one or more substrates selected from the group consisting of amylose, arabinan, arabinoxylan, casein and xylan e.g. determined as described in Example 1.

The invention relates in a preferred embodiment to a composition such as an animal feed composition (such as e.g. a swine or poultry feed composition) comprising spores of a Bacillus strain wherein:

i. at least 60% (such as at least 65%, at least 70%, at least 80% or at least 90%) of the Bacillus spores survive gastric stability e.g. determined as described in Example 1; and/or

ii. at least 55% (such as at least 60%, at least 70%, at least 80% or at least 90%) of the Bacillus spores germinate in less than 4 hours e.g. determined as described in Example 1,

iii. the Bacillus strain is selected from the group consisting of:
the strain having the deposit accession number NRRL B-50136;
the strain having the deposit accession number NRRL B-50147;
the strain having the deposit accession number NRRL B-50141;
the strain having the deposit accession number PTA-7543;
the strain having the deposit accession number NRRL B-50888;
the strain having the deposit accession number PTA-7549; and
the strain having the deposit accession number NRRL B-50349 (or a strain having all of the identifying characteristics of any of the strains or a mutant of any of the strains), or any combination thereof, and

iv. the Bacillus spores have antimicrobial activity against Clostridium perfringens e.g. as determined in Example 1.

In one embodiment, the invention relates to a composition such as an animal feed composition (such as e.g. a swine or poultry feed composition) comprising spores of a Bacillus strain wherein:

i. at least 60% (such as at least 65%, at least 70%, at least 80% or at least 90%) of the Bacillus spores survive gastric stability e.g. as determined in Example 1; and/or

ii. at least 55% (such as at least 60%, at least 70%, at least 80% or at least 90%) of the Bacillus spores germinate in less than 4 hours e.g. as determined in Example 1,

iii. the Bacillus strain is selected from the group consisting of:
the strain having the deposit accession number NRRL B-50136;
the strain having the deposit accession number NRRL B-50147;
the strain having the deposit accession number NRRL B-50141;
the strain having the deposit accession number PTA-7543;
the strain having the deposit accession number NRRL B-50888;
the strain having the deposit accession number PTA-7549; and
the strain having the deposit accession number NRRL B-50349 (or a strain having all of the identifying characteristics of any of the strains or a mutant of any of the strains), or any combination thereof, and

iv. the Bacillus spores have antimicrobial activity against Clostridium perfringens e.g. as determined in Example 1 and wherein the composition is for treatment of a Clostridium perfringens infection in an animal such as pig or poultry.

In one embodiment, the invention relates to a composition such as an animal feed composition (such as e.g. a swine or poultry feed composition) comprising spores of a Bacillus strain wherein:

i. at least 60% (such as at least 65%, at least 70%, at least 80% or at least 90%) of the Bacillus spores survive gastric stability e.g. as determined in Example 1; and/or

ii. at least 55% (such as at least 60%, at least 70%, at least 80% or at least 90%) of the Bacillus spores germinate in less than 4 hours e.g. as determined in Example 1,

iii. the Bacillus strain is selected from the group consisting of:
the strain having the deposit accession number NRRL B-50136;
the strain having the deposit accession number NRRL B-50147;
the strain having the deposit accession number NRRL B-50141;
the strain having the deposit accession number PTA-7543;
the strain having the deposit accession number NRRL B-50888;
the strain having the deposit accession number PTA-7549; and
the strain having the deposit accession number NRRL B-50349 (or a strain having all of the identifying characteristics of any of the strains or a mutant of any of the strains), or any combination thereof, and

iv. the Bacillus spores have antimicrobial activity against Clostridium perfringens e.g. determined as described in Example 1 and

wherein the composition is for treatment of necrotic enteritis in an animal such as pig or poultry.

In one embodiment the animal is not a human being. In a further embodiment, the animal is a mono-gastric animal. Mono-gastric animals include, but are not limited to, pigs or swine (including, but not limited to, piglets, growing pigs, and sows); poultry such as turkeys, ducks and chicken (including but not limited to broilers, chicks, layers); horses (including but not limited to hotbloods, coldbloods and warm bloods) and fish (including but not limited to salmon, trout, tilapia, catfish and carps; and crustaceans (including but not limited to shrimps and prawns). Pigs and/or poultry are preferred mono-gastric animals.

The animal feed can further comprise one or more components selected from the list consisting of concentrate; forage; one or more enzymes; one or more additional microbes; one or more vitamins; one or more minerals; one or more amino acids; and one or more other feed ingredients.

In a further embodiment, the composition such as the animal feed comprises one or more bacterial strains such as at least two of the above strains, at least three of the above strains, at least four of the above strains, at least five of the above strains, at least six of the above strains, up to and including all of the above strains.

In one embodiment, the composition such as the animal feed comprises the Bacillus strains having the deposit accession numbers NRRL B-50136 and NRRL B-50147. In one embodiment, the composition such as the animal feed comprises the Bacillus strains having the deposit accession numbers NRRL B-50136 and NRRL B-50141. In one embodiment, the composition such as the animal feed comprises the Bacillus strains having the deposit accession numbers NRRL B-50136 and PTA-7543. In one embodiment, the composition such as the animal feed comprises the Bacillus strains having the deposit accession numbers NRRL B-50136 and NRRL B-50888. In one embodiment, the composition such as the animal feed comprises the Bacillus strains having the deposit accession numbers NRRL B-50136 and PTA-7549. In one embodiment, the composition such as the animal feed comprises the Bacillus strains having the deposit accession numbers NRRL B-50136 and NRRL B-50349.

In one embodiment, the composition such as the animal feed comprises the Bacillus strains having the deposit accession numbers NRRL B-50147 and NRRL B-50141. In one embodiment, the composition such as the animal feed comprises the Bacillus strains having the deposit accession numbers NRRL B-50147 and PTA-7543. In one embodiment, the composition such as the animal feed comprises the Bacillus strains having the deposit accession numbers NRRL B-50147 and NRRL B-50888. In one embodiment, the composition such as the animal feed comprises the Bacillus strains having the deposit accession numbers NRRL B-50147 and PTA-7549. In one embodiment, the composition such as the animal feed comprises the Bacillus strains having the deposit accession numbers NRRL B-50147 and NRRL B-50349.

In one embodiment, the composition such as the animal feed comprises the Bacillus strains having the deposit accession numbers NRRL B-50141 and PTA-7543. In one embodiment, the composition such as the animal feed comprises the Bacillus strains having the deposit accession numbers NRRL B-50141 and NRRL B-50888. In one embodiment, the composition such as the animal feed comprises the Bacillus strains having the deposit accession numbers NRRL B-50141 and PTA-7549. In one embodiment, the composition such as the animal feed comprises the Bacillus strains having the deposit accession numbers NRRL B-50141 and NRRL B-50349.

In one embodiment, the composition such as the animal feed comprises the Bacillus strains having the deposit accession numbers PTA-7543 and NRRL B-50888. In one embodiment, the composition such as the animal feed comprises the Bacillus strains having the deposit accession numbers PTA-7543 and PTA-7549. In one embodiment, the composition such as the animal feed comprises the Bacillus strains having the deposit accession numbers PTA-7543 and NRRL B-50349.

In one embodiment, the composition such as the animal feed comprises the Bacillus strains having the deposit accession numbers NRRL B-50888 and PTA-7549. In one embodiment, the composition such as the animal feed comprises the Bacillus strains having the deposit accession numbers NRRL B-50888 and NRRL B-50349. In one embodiment, the composition such as the animal feed comprises the Bacillus strains having the deposit accession numbers PTA-7549 and NRRL B-50349.

In a preferred embodiment, the invention relates to a composition comprising spores of a Bacillus strain wherein:

i. at least 60% (such as at least 65%) of the Bacillus spores survive gastric stability e.g. determined as described in Example 1; and/or

ii. at least 55% of the Bacillus spores germinate in less than 4 hours e.g. determined as described in Example 1,

iii. the Bacillus strain has the deposit accession number NRRL B-50136 (or a strain having all of the identifying characteristics of the strain or a mutant of the strain) and

iv. the Bacillus spores have antimicrobial activity against Clostridium perfringens e.g. determined as described in Example 1.

In a preferred embodiment, the invention relates to a composition comprising spores of a Bacillus strain wherein:

i. at least 60% (such as at least 65%, 70%, 80% or 90%) of the Bacillus spores survive gastric stability e.g. determined as described in Example 1; and/or

ii. at least 55% (such as at least 60%, 70%, 80% or 90%) of the Bacillus spores germinate in less than 4 hours e.g. determined as described in Example 1,

iii. the Bacillus strain has the deposit accession number NRRL B-50147 (or a strain having all of the identifying characteristics of the strain or a mutant of the strain) and

iv. the Bacillus spores have antimicrobial activity against Clostridium perfringens e.g. determined as described in Example 1.

In a preferred embodiment, the invention relates to a composition comprising spores of a Bacillus strain wherein:

i. at least 60% (such as at least 65%, 70%, 80% or 90%) of the Bacillus spores survive gastric stability e.g. determined as described in Example 1; and/or

ii. at least 55% (such as at least 60%, 70%, 80% or 85%) of the Bacillus spores germinate in less than 4 hours e.g. determined as described in Example 1,

iii. the Bacillus strain has the deposit accession number NRRL B-50141 (or a strain having all of the identifying characteristics of the strain or a mutant of the strain) and

iv. the Bacillus spores have antimicrobial activity against Clostridium perfringens e.g. determined as described in Example 1.

In a preferred embodiment, the invention relates to a composition comprising spores of a Bacillus strain wherein:

i. at least 60% (such as at least 65%, 70%, 80% or 90%) of the Bacillus spores survive gastric stability e.g. determined as described in Example 1; and/or

ii. at least 55% (such as at least 60%, 70%, 80% or 90%) of the Bacillus spores germinate in less than 4 hours e.g. determined as described in Example 1,

iii. the Bacillus strain has the deposit accession number PTA-7543 (or a strain having all of the identifying characteristics of the strain or a mutant of the strain) and

iv. the Bacillus spores have antimicrobial activity against Clostridium perfringens e.g. determined as described in Example 1.

In a preferred embodiment, the invention relates to a composition comprising spores of a Bacillus strain wherein:

i. at least 60% (such as at least 65%, 70%, or 75%) of the Bacillus spores survive gastric stability e.g. determined as described in Example 1; and/or

ii. at least 55% (such as at least 60%, 70%, 80% or 90%) of the Bacillus spores germinate in less than 4 hours e.g. determined as described in Example 1,

iii. the Bacillus strain has the deposit accession number NRRL B-50888 (or a strain having all of the identifying characteristics of the strain or a mutant of the strain) and

iv. the Bacillus spores have antimicrobial activity against Clostridium perfringens e.g. determined as described in Example 1.

In a preferred embodiment, the invention relates to a composition comprising spores of a Bacillus strain wherein:

i. at least 60% (such as at least 65%, 70%, 80% or 90%) of the Bacillus spores survive gastric stability e.g. determined as described in Example 1; and/or

ii. at least 55% (such as at least 60%, 70%, 80% or 85%) of the Bacillus spores germinate in less than 4 hours e.g. determined as described in Example 1,

iii. the Bacillus strain has the deposit accession number PTA-7549 (or a strain having all of the identifying characteristics of the strain or a mutant of the strain) and

iv. the Bacillus spores have antimicrobial activity against Clostridium perfringens e.g. determined as described in Example 1.

In a preferred embodiment, the invention relates to a composition comprising spores of a Bacillus strain wherein:

i. at least 60% (such as at least 62%) of the Bacillus spores survive gastric stability e.g. determined as described in Example 1; and/or

ii. at least 55% (such as at least 60%, 70%, 80% or 90%) of the Bacillus spores germinate in less than 4 hours e.g. determined as described in Example 1,

iii. the Bacillus strain has the deposit accession number NRRL B-50349 (or a strain having all of the identifying characteristics of the strain or a mutant of the strain) and

iv. the Bacillus spores have antimicrobial activity against Clostridium perfringens e.g. determined as described in Example 1.

In a preferred embodiment, the invention relates to a composition for treatment of C. perfringens infections in a mono-gastric animal, wherein the composition comprises spores of a Bacillus strain wherein:

i. at least 60% (such as at least 65%) of the Bacillus spores survive gastric stability e.g. determined as described in Example 1; and/or

ii. at least 55% of the Bacillus spores germinate in less than 4 hours e.g. determined as described in Example 1,

iii. the Bacillus strain has the deposit accession number NRRL B-50136 (or a strain having all of the identifying characteristics of the strain or a mutant of the strain) and

iv. the Bacillus spores have antimicrobial activity against Clostridium perfringens e.g. determined as described in Example 1.

In a preferred embodiment, the invention relates to a composition for treatment of C. perfringens infections in a mono-gastric animal, wherein the composition comprises spores of a Bacillus strain wherein:

i. at least 60% (such as at least 65%, 70%, 80% or 90%) of the Bacillus spores survive gastric stability e.g. determined as described in Example 1; and/or

ii. at least 55% (such as at least 60%, 70%, 80% or 90%) of the Bacillus spores germinate in less than 4 hours e.g. determined as described in Example 1,

iii. the Bacillus strain has the deposit accession number NRRL B-50147 (or a strain having all of the identifying characteristics of the strain or a mutant of the strain) and

iv. the Bacillus spores have antimicrobial activity against Clostridium perfringens e.g. determined as described in Example 1.

In a preferred embodiment, the invention relates to a composition for treatment of C. perfringens infections in a mono-gastric animal, wherein the composition comprises spores of a Bacillus strain wherein:

i. at least 60% (such as at least 65%, 70%, 80% or 90%) of the Bacillus spores survive gastric stability e.g. determined as described in Example 1; and/or

ii. at least 55% (such as at least 60%, 70%, 80% or 85%) of the Bacillus spores germinate in less than 4 hours e.g. determined as described in Example 1,

iii. the Bacillus strain has the deposit accession number NRRL B-50141 (or a strain having all of the identifying characteristics of the strain or a mutant of the strain) and

iv. the Bacillus spores have antimicrobial activity against Clostridium perfringens e.g. determined as described in Example 1.

In a preferred embodiment, the invention relates to a composition for treatment of C. perfringens infections in a mono-gastric animal, wherein the composition comprises spores of a Bacillus strain wherein:

i. at least 60% (such as at least 65%, 70%, 80% or 90%) of the Bacillus spores survive gastric stability e.g. determined as described in Example 1; and/or

ii. at least 55% (such as at least 60%, 70%, 80% or 90%) of the Bacillus spores germinate in less than 4 hours e.g. determined as described in Example 1,

iii. the Bacillus strain has the deposit accession number PTA-7543 (or a strain having all of the identifying characteristics of the strain or a mutant of the strain) and

iv. the Bacillus spores have antimicrobial activity against Clostridium perfringens e.g. determined as described in Example 1.

In a preferred embodiment, the invention relates to a composition for treatment of C. perfringens infections in a mono-gastric animal, wherein the composition comprises spores of a Bacillus strain wherein:

i. at least 60% (such as at least 65%, 70%, or 75%) of the Bacillus spores survive gastric stability e.g. determined as described in Example 1; and/or

ii. at least 55% (such as at least 60%, 70%, 80% or 90%) of the Bacillus spores germinate in less than 4 hours e.g. determined as described in Example 1,

iii. the Bacillus strain has the deposit accession number NRRL B-50888 (or a strain having all of the identifying characteristics of the strain or a mutant of the strain) and

iv. the Bacillus spores have antimicrobial activity against Clostridium perfringens e.g. determined as described in Example 1.

In a preferred embodiment, the invention relates to a composition for treatment of C. perfringens infections in a mono-gastric animal, wherein the composition comprises spores of a Bacillus strain wherein:

i. at least 60% (such as at least 65%, 70%, 80% or 90%) of the Bacillus spores survive gastric stability e.g. determined as described in Example 1; and/or

ii. at least 55% (such as at least 60%, 70%, 80% or 85%) of the Bacillus spores germinate in less than 4 hours e.g. determined as described in Example 1,

iii. the Bacillus strain has the deposit accession number PTA-7549 (or a strain having all of the identifying characteristics of the strain or a mutant of the strain) and

iv. the Bacillus spores have antimicrobial activity against Clostridium perfringens e.g. determined as described in Example 1.

In a preferred embodiment, the invention relates to a composition for treatment of C. perfringens infections in a mono-gastric animal, wherein the composition comprises spores of a Bacillus strain wherein:

i. at least 60% (such as at least 62%) of the Bacillus spores survive gastric stability e.g. determined as described in Example 1; and/or

ii. at least 55% (such as at least 60%, 70%, 80% or 90%) of the Bacillus spores germinate in less than 4 hours e.g. determined as described in Example 1,

iii. the Bacillus strain has the deposit accession number NRRL B-50349 (or a strain having all of the identifying characteristics of the strain or a mutant of the strain) and

iv. the Bacillus spores have antimicrobial activity against Clostridium perfringens e.g. determined as described in Example 1.

In a preferred embodiment, the invention relates to a composition for treatment of necrotic enteritis in a mono-gastric animal, wherein the composition comprises spores of a Bacillus strain wherein:

i. at least 60% (such as at least 65%) of the Bacillus spores survive gastric stability e.g. determined as described in Example 1; and/or

ii. at least 55% of the Bacillus spores germinate in less than 4 hours e.g. determined as described in Example 1,

iii. the Bacillus strain has the deposit accession number NRRL B-50136 (or a strain having all of the identifying characteristics of the strain or a mutant of the strain) and

iv. the Bacillus spores have antimicrobial activity against Clostridium perfringens e.g. determined as described in Example 1.

In a preferred embodiment, the invention relates to a composition for treatment of necrotic enteritis in a mono-gastric animal, wherein the composition comprises spores of a Bacillus strain wherein:

i. at least 60% (such as at least 65%, 70%, 80% or 90%) of the Bacillus spores survive gastric stability e.g. determined as described in Example 1; and/or

ii. at least 55% (such as at least 60%, 70%, 80% or 90%) of the Bacillus spores germinate in less than 4 hours e.g. determined as described in Example 1,

iii. the Bacillus strain has the deposit accession number NRRL B-50147 (or a strain having all of the identifying characteristics of the strain or a mutant of the strain) and

iv. the Bacillus spores have antimicrobial activity against Clostridium perfringens e.g. determined as described in Example 1.

In a preferred embodiment, the invention relates to a composition for treatment of necrotic enteritis in a mono-gastric animal, wherein the composition comprises spores of a Bacillus strain wherein:

i. at least 60% (such as at least 65%, 70%, 80% or 90%) of the Bacillus spores survive gastric stability e.g. determined as described in Example 1; and/or

ii. at least 55% (such as at least 60%, 70%, 80% or 85%) of the Bacillus spores germinate in less than 4 hours e.g. determined as described in Example 1,

iii. the Bacillus strain has the deposit accession number NRRL B-50141 (or a strain having all of the identifying characteristics of the strain or a mutant of the strain) and

iv. the Bacillus spores have antimicrobial activity against Clostridium perfringens e.g. determined as described in Example 1.

In a preferred embodiment, the invention relates to a composition for treatment of necrotic enteritis in a mono-gastric animal, wherein the composition comprises spores of a Bacillus strain wherein:

i. at least 60% (such as at least 65%, 70%, 80% or 90%) of the Bacillus spores survive gastric stability e.g. determined as described in Example 1; and/or

ii. at least 55% (such as at least 60%, 70%, 80% or 90%) of the Bacillus spores germinate in less than 4 hours e.g. determined as described in Example 1,

iii. the Bacillus strain has the deposit accession number PTA-7543 (or a strain having all of the identifying characteristics of the strain or a mutant of the strain) and

iv. the Bacillus spores have antimicrobial activity against Clostridium perfringens e.g. determined as described in Example 1.

In a preferred embodiment, the invention relates to a composition for treatment of necrotic enteritis in a mono-gastric animal, wherein the composition comprises spores of a Bacillus strain wherein:

i. at least 60% (such as at least 65%, 70%, or 75%) of the Bacillus spores survive gastric stability e.g. determined as described in Example 1; and/or

ii. at least 55% (such as at least 60%, 70%, 80% or 90%) of the Bacillus spores germinate in less than 4 hours e.g. determined as described in Example 1,

iii. the Bacillus strain has the deposit accession number NRRL B-50888 (or a strain having all of the identifying characteristics of the strain or a mutant of the strain) and

iv. the Bacillus spores have antimicrobial activity against Clostridium perfringens e.g. determined as described in Example 1.

In a preferred embodiment, the invention relates to a composition for treatment of necrotic enteritis in a mono-gastric animal, wherein the composition comprises spores of a Bacillus strain wherein:

i. at least 60% (such as at least 65%, 70%, 80% or 90%) of the Bacillus spores survive gastric stability e.g. determined as described in Example 1; and/or

ii. at least 55% (such as at least 60%, 70%, 80% or 85%) of the Bacillus spores germinate in less than 4 hours e.g. determined as described in Example 1,

iii. the Bacillus strain has the deposit accession number PTA-7549 (or a strain having all of the identifying characteristics of the strain or a mutant of the strain) and

iv. the Bacillus spores have antimicrobial activity against Clostridium perfringens e.g. determined as described in Example 1.

In a preferred embodiment, the invention relates to a composition for treatment of necrotic enteritis in a mono-gastric animal, wherein the composition comprises spores of a Bacillus strain wherein:

i. at least 60% (such as at least 62%) of the Bacillus spores survive gastric stability e.g. determined as described in Example 1; and/or

ii. at least 55% (such as at least 60%, 70%, 80% or 90%) of the Bacillus spores germinate in less than 4 hours e.g. determined as described in Example 1,

iii. the Bacillus strain has the deposit accession number NRRL B-50349 (or a strain having all of the identifying characteristics of the strain or a mutant of the strain) and

iv. the Bacillus spores have antimicrobial activity against Clostridium perfringens e.g. determined as described in Example 1.

In an embodiment to any of the aforementioned embodiments the Bacillus spore kills/inhibits at least 40% (such as at least 45%, at least 50%, at least 60%, at least 70% or at least 80%) of Clostridium perfringens after 24 hours e.g. determined as described in Example 1.

In an embodiment to any of the aforementioned embodiments wherein at least 60% (such as at least 65%, 70%, 80%, 90% or 95%) of the Bacillus spores survive gastric stability e.g. determined as described in Example 1.

In an embodiment to any of the aforementioned embodiments wherein at least 55% (such as at least 60%, 70%, 80%, 90% or 95%) of the Bacillus spores germinate in less than 4 hours e.g. determined as described in Example 1.

In another embodiment the invention the composition such as the animal feed further comprises concentrate. In another embodiment of the invention the composition such as the animal feed further comprises forage. In another embodiment of the invention the composition such as the animal feed further comprises one or more additional microbes. In another embodiment of the invention the composition such as the animal feed further comprises one or more enzymes. In another embodiment of the invention the composition such as the animal feed further comprises one or more vitamins. In another embodiment of the invention the composition such as the animal feed further comprises one or more minerals. In another embodiment of the invention the composition such as the animal feed further comprises one or more amino acids. In another embodiment of the invention the composition such as the animal feed further comprises one or more other feed ingredients.

In an embodiment to any of the aforementioned embodiments, the composition also improves the health of the mono-gastric animal when fed to the animal. In another embodiment to any of the aforementioned embodiments, the composition also increases the egg yield of poultry when fed the poultry. In an embodiment to any of the aforementioned embodiments, the composition increases the meat yield of the mono-gastric animal when fed to the animal.

In a preferred embodiment, the composition such as the animal feed (e.g. poultry or swine feed) comprises one or more bacterial strains described herein, wherein the bacterial count of each of the bacterial strains is between 1×10⁴ and 1×10¹⁴ CFU/kg of composition, preferably between 1×10⁶ and 1×10¹² CFU/kg of composition, and more preferably between 1×10⁷ and 1×10¹¹ CFU/kg of composition. In a more preferred embodiment the bacterial count of each of the bacterial strains described herein is between 1×10⁸ and 1×10¹⁰ CFU/kg of composition.

In a preferred embodiment, the bacterial count of each of the bacterial strains in the animal feed composition (e.g. poultry or swine feed) is between 1×10⁴ and 1×10¹⁴ CFU/kg of dry matter, preferably between 1×10⁶ and 1×10¹² CFU/kg of dry matter, and more preferably between 1×10⁷ and 1×10¹¹ CFU/kg of dry matter. In a more preferred embodiment the bacterial count of each of the bacterial strains in the animal feed composition is between 1×10⁸ and 1×10¹⁰CFU/kg of dry matter.

In a preferred embodiment, the composition such as the animal feed (e.g. poultry or swine feed) has a bacterial count of each Bacillus spore between 1×10⁵ and 1×10¹⁵ CFU/animal/day, preferably between 1×10⁷ and 1×10¹³ CFU/animal/day, and more preferably between 1×10⁸ and 1×10¹² CFU/animal/day.

In still yet another embodiment of the invention, the one or more bacterial strains are present in the composition in form of a spore such as a stable spore. In still a further embodiment of the invention, the stable spore will germinate in the intestine and/or stomach of the mono-gastric animal.

In one embodiment, the one or more bacterial strains are stable when subjected to pressures applied/achieved during an extrusion process for pelleting. In a particular embodiment, the one or more bacterial strains are stable at pressures ranging from 1 bar to 40 bar, particularly 10 bar to 40 bar, more particularly 15 bar to 40 bar, even more particularly 20 bar to 40 bar, still even more particularly 35 bar to 37 bar, even still more particularly 36 bar.

In a particular embodiment, the one or more bacterial strains are stable at high temperatures. In particular, the bacterial strains are stable when they are subjected to temperatures achieved during an extrusion process for pelleting. In an even more particular embodiment, the one or more bacterial strains are stable at temperatures ranging from 80° C. to 120° C., particularly temperatures ranging from, 90° C. to 120° C., even more particularly temperatures ranging from 95° C. to 120° C.

In another aspect, the invention relates to a composition such as an animal feed composition comprising a carrier, such as forage and one or more of the bacteria cultures having characteristics substantially identical to that of a strain selected from the group consisting of:

the strain having the deposit accession number NRRL B-50136;

the strain having the deposit accession number NRRL B-50147;

the strain having the deposit accession number NRRL B-50141;

the strain having the deposit accession number PTA-7543;

the strain having the deposit accession number NRRL B-50888;

the strain having the deposit accession number PTA-7549; and

the strain having the deposit accession number NRRL B-50349, or any combination thereof.

In another aspect, the invention relates to a composition such as an animal feed composition comprising a carrier, such as forage and one or more of mutants of a strain selected from the group consisting of:

the strain having the deposit accession number NRRL B-50136;

the strain having the deposit accession number NRRL B-50147;

the strain having the deposit accession number NRRL B-50141;

the strain having the deposit accession number PTA-7543;

the strain having the deposit accession number NRRL B-50888;

the strain having the deposit accession number PTA-7549; and

the strain having the deposit accession number NRRL B-50349, or any combination thereof.

In an embodiment, the animal feed composition comprises a carrier and the strain having the deposit accession number NRRL B-50136 (or a strain having all of the identifying characteristics of the strain or a mutant of the strain).

In an embodiment, the animal feed composition comprises a carrier and the strain having the deposit accession number NRRL B-50147 (or a strain having all of the identifying characteristics of the strain or a mutant of the strain).

In an embodiment, the animal feed composition comprises a carrier and the strain having the deposit accession number NRRL B-50141 (or a strain having all of the identifying characteristics of the strain or a mutant of the strain).

In an embodiment, the animal feed composition comprises a carrier and the strain having the deposit accession number PTA-7543 (or a strain having all of the identifying characteristics of the strain or a mutant of the strain).

In an embodiment, the animal feed composition comprises a carrier and the strain having the deposit accession number NRRL B-50888 (or a strain having all of the identifying characteristics of the strain or a mutant of the strain).

In an embodiment, the animal feed composition comprises a carrier and the strain having the deposit accession number PTA-7549 (or a strain having all of the identifying characteristics of the strain or a mutant of the strain).

In an embodiment, the animal feed composition comprises a carrier and the strain having the deposit accession number NRRL B-50349 (or a strain having all of the identifying characteristics of the strain or a mutant of the strain).

In another embodiment, the animal feed composition is for feeding to a mono-gastric animal. Mono-gastric animals include, but are not limited to, pigs or swine (including, but not limited to, piglets, growing pigs, and sows); poultry such as turkeys, ducks and chicken (including but not limited to broiler chicks, layers); horses (including but not limited to hotbloods, coldbloods and warm bloods) and fish (including but not limited to salmon, trout, tilapia, catfish and carps; and crustaceans (including but not limited to shrimps and prawns). Pigs and/or poultry are preferred mono-gastric animals.

In an embodiment, the animal feed composition further comprises one or more additional microbes. In a particular embodiment, the animal feed composition further comprises a bacterium from one or more of the following genera: Lactobacillus, Lactococcus, Streptococcus, Bacillus, Pediococcus, Enterococcus, Leuconostoc, Carnobacterium, Propionibacterium, Bifidobacterium, Clostridium and Megasphaera or any combination thereof.

In a particular embodiment, animal feed composition further comprises a bacterium from one or more of the following strains of Bacillus amyloliquefaciens, Bacillus subtilis, Bacillus pumilus, Bacillus polymyxa, Bacillus licheniformis, Bacillus megaterium, Bacillus coagulans, Bacillus circulans, or any combination thereof.

In a particular embodiment, the animal feed composition further comprises one or more types of yeast. The one or more types of yeast can be selected from the group consisting of Saccharomycetaceae, Saccharomyces (such as S. cerevisiae and/or S. boulardii), Kluyveromyces (such as K. marxianus and K. lactis), Candida (such as C. utilis, also called Torula yeast), Pichia (such as P. pastoris), Torulaspora (such as T. delbrueckii), Phaffia yeasts and Basidiomycota.

In a more particular embodiment, the animal feed composition further comprises one or more bacterium from one or more Bacillus strains selected from the group consisting of:

the strain having the deposit accession number NRRL B-50136 (or a strain having all of the identifying characteristics of the strain or a mutant of the strain);

the strain having the deposit accession number NRRL B-50147 (or a strain having all of the identifying characteristics of the strain or a mutant of the strain);

the strain having the deposit accession number NRRL B-50141 (or a strain having all of the identifying characteristics of the strain or a mutant of the strain);

the strain having the deposit accession number PTA-7543 (or a strain having all of the identifying characteristics of the strain or a mutant of the strain);

the strain having the deposit accession number NRRL B-50888 (or a strain having all of the identifying characteristics of the strain or a mutant of the strain);

the strain having the deposit accession number PTA-7549 (or a strain having all of the identifying characteristics of the strain or a mutant of the strain); and

the strain having the deposit accession number NRRL B-50349 (or a strain having all of the identifying characteristics of the strain or a mutant of the strain), or any combination thereof, and such as, at least three of the above strains, at least four of the above strains, at least five of the above strains, at least six of the above strains up to and including all of the above strains.

In an embodiment to any of the aforementioned embodiments the composition further comprises a formulating agent. The formulating agent can comprise one or more of the following compounds: glycerol, ethylene glycol, 1, 2-propylene glycol, 1, 3-propylene glycol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch and cellulose.

### Animal Feed

In one aspect, the animal feed comprises concentrates as well as vitamins, minerals, enzymes, amino acids and/or other feed ingredients (such as in a premix). In a specific embodiment the animal feed further comprises forage.

Forage as defined herein also includes roughage. Forage is fresh plant material such as hay and silage from forage plants, grass and other forage plants, grass and other forage plants, seaweed, sprouted grains and legumes, or any combination thereof. Examples of forage plants are Alfalfa (lucerne), birdsfoot trefoil, brassica (e.g. kale, rapeseed (canola), rutabaga (swede), turnip), clover (e.g. alsike clover, red clover, subterranean clover, white clover), grass (e.g. Bermuda grass, brome, false oat grass, fescue, heath grass, meadow grasses, orchard grass, ryegrass, Timothy-grass), corn (maize), millet, barley, oats, rye, sorghum, soybeans and wheat and vegetables such as beets. Crops suitable for ensilage are the ordinary grasses, clovers, alfalfa, vetches, oats, rye and maize. Forage further includes crop residues from grain production (such as corn stover; straw from wheat, barley, oat, rye and other grains); residues from vegetables like beet tops; residues from oilseed production like stems and leaves form soy beans, rapeseed and other legumes; and fractions from the refining of grains for animal or human consumption or from fuel production or other industries.

Roughage is generally dry plant material with high levels of fiber, such as fiber, bran, husks from seeds and grains and crop residues (such as stover, copra, straw, chaff, sugar beet waste).

Examples of concentrates are feed with high protein and energy concentrations, such as fish meal, molasses, oligosaccharides, sorghum, seeds and grains (either whole or prepared by crushing, milling, etc. from e.g. corn, oats, rye, barley, wheat), oilseed press cake (e.g. from cottonseed, safflower, sunflower, soybean, rapeseed/canola, peanut or groundnut), palm kernel cake, yeast derived material and distillers grains (such as wet distillers grains (WDS) and dried distillers grains with solubles (DDGS)).

In one embodiment, the forage and one or more microbes are mixed with a concentrate. In another embodiment, the forage and one or more microbes are mixed with a premix. In a further embodiment, the forage and one or more microbes are mixed with vitamins and/or minerals. In a further embodiment, the forage and one or more microbes are mixed with one or more enzymes. In a further embodiment, the forage and one or more microbes are mixed with other feed ingredients, such as colouring agents, stabilisers, growth improving additives and aroma compounds/flavorings, polyunsaturated fatty acids (PUFAs); reactive oxygen generating species, anti-microbial peptides, anti-fungal polypeptides and amino acids.

In particular embodiments, the animal feed may comprise 0-80% maize; and/or 0-80% sorghum; and/or 0-70% wheat; and/or 0-70% barley; and/or 0-30% oats; and/or 0-40% soybean meal; and/or 0-10% fish meal; and/or 0-20% whey.

The animal feed may comprise vegetable proteins. In particular embodiments, the protein content of the vegetable proteins is at least 10, 20, 30, 40, 50, 60, 70, 80, or 90% (w/w). Vegetable proteins may be derived from vegetable protein sources, such as legumes and cereals, for example, materials from plants of the families Fabaceae (Leguminosae), Cruciferaceae, Chenopodiaceae, and Poaceae, such as soy bean meal, lupin meal, rapeseed meal, and combinations thereof.

In a particular embodiment, the vegetable protein source is material from one or more plants of the family Fabaceae, e.g., soybean, lupine, pea, or bean. In another particular embodiment, the vegetable protein source is material from one or more plants of the family Chenopodiaceae, e.g. beet, sugar beet, spinach or quinoa. Other examples of vegetable protein sources are rapeseed, and cabbage. In another particular embodiment, soybean is a preferred vegetable protein source. Other examples of vegetable protein sources are cereals such as barley, wheat, rye, oat, maize (corn), rice, and sorghum.

In a particular embodiment the animal feed consists of or comprises milk (e.g. from sow) e.g. for feeding of piglets. In another particular embodiment the animal feed consists of or comprises milk replacement e.g. for feeding of piglets.

### Premix

In an embodiment, the animal feed may include a premix, comprising e.g. vitamins, minerals, enzymes, preservatives, antibiotics, other feed ingredients or any combination thereof which are mixed into the animal feed.

### Vitamins and Minerals

In another embodiment, the animal feed may include one or more vitamins, such as one or more fat-soluble vitamins and/or one or more water-soluble vitamins. In another embodiment, the animal feed may optionally include one or more minerals, such as one or more trace minerals and/or one or more macro minerals.

Usually fat- and water-soluble vitamins, as well as trace minerals form part of a so-called premix intended for addition to the feed, whereas macro minerals are usually separately added to the feed.

Non-limiting examples of fat-soluble vitamins include vitamin A, vitamin D3, vitamin E, and vitamin K, e.g., vitamin K3.

Non-limiting examples of water-soluble vitamins include vitamin B12, biotin and choline, vitamin B1, vitamin B2, vitamin B6, niacin, folic acid and panthothenate, e.g., Ca-D-panthothenate.

Non-limiting examples of trace minerals include boron, cobalt, chloride, chromium, copper, fluoride, iodine, iron, manganese, molybdenum, selenium and zinc.

Non-limiting examples of macro minerals include calcium, magnesium, potassium and sodium.

### Enzymes

In another embodiment, the animal feed compositions described herein optionally include one or more enzymes. Enzymes can be classified on the basis of the handbook Enzyme Nomenclature from NC-IUBMB, 1992), see also the ENZYME site at the internet: www.expasy.ch/enzyme/. ENZYME is a repository of information relative to the nomenclature of enzymes. It is primarily based on the recommendations of the Nomenclature Committee of the International Union of Biochemistry and Molecular Biology (IUB-MB), Academic Press, Inc., 1992, and it describes each type of characterized enzyme for which an EC (Enzyme Commission) number has been provided (Bairoch A. The ENZYME database, 2000, Nucleic Acids Res 28:304-305). This IUB-MB Enzyme nomenclature is based on their substrate specificity and occasionally on their molecular mechanism; such a classification does not reflect the structural features of these enzymes.

Another classification of certain glycoside hydrolase enzymes, such as endoglucanase, xylanase, galactanase, mannanase, dextranase and alpha-galactosidase, in families based on amino acid sequence similarities has been proposed a few years ago. They currently fall into 90 different families: See the CAZy(ModO) internet site (Coutinho, P. M. & Henrissat, B. (1999) Carbohydrate-Active Enzymes server at URL: afmb.cnrs-mrs.fr/˜cazy/CAZY/index.html (corresponding papers: Coutinho, P. M. & Henrissat, B. (1999) Carbohydrate-active enzymes: an integrated database approach. In “Recent Advances in Carbohydrate Bioengineering”, H. J. Gilbert, G. Davies, B. Henrissat and B. Svensson eds., The Royal Society of Chemistry, Cambridge, pp. 3-12; Coutinho, P. M. & Henrissat, B. (1999) The modular structure of cellulases and other carbohydrate-active enzymes: an integrated database approach. In “Genetics, Biochemistry and Ecology of Cellulose Degradation”., K. Ohmiya, K. Hayashi, K. Sakka, Y. Kobayashi, S. Karita and T. Kimura eds., Uni Publishers Co., Tokyo, pp. 15-23).

Thus the composition of the invention may also comprise at least one other enzyme selected from the group comprising of phytase (EC 3.1.3.8 or 3.1.3.26); xylanase (EC 3.2.1.8); galactanase (EC 3.2.1.89); alpha-galactosidase (EC 3.2.1.22); protease (EC 3.4); phospholipase A1 (EC 3.1.1.32); phospholipase A2 (EC 3.1.1.4); lysophospholipase (EC 3.1.1.5); phospholipase C (3.1.4.3); phospholipase D (EC 3.1.4.4); amylase such as, for example, alpha-amylase (EC 3.2.1.1); lysozyme (EC 3.2.1.17); and beta-glucanase (EC 3.2.1.4 or EC 3.2.1.6), or any mixture thereof.

In a particular embodiment, the composition of the invention comprises a phytase (EC 3.1.3.8 or 3.1.3.26). Examples of commercially available phytases include Bio-Feed™ Phytase (Novozymes), Ronozyme® P and HiPhos™ (DSM Nutritional Products), Natuphos™ (BASF), Finase® and Quantum® Blue (AB Enzymes), the Phyzyme® XP (Verenium/DuPont) and Axtra® PHY (DuPont). Other preferred phytases include those described in e.g. WO 98/28408, WO 00/43503, and WO 03/066847.

In a particular embodiment, the composition of the invention comprises a xylanase (EC 3.2.1.8). Examples of commercially available xylanases include Ronozyme® WX and G2 (DSM Nutritional Products), Econase® XT and Barley (AB Vista), Xylathin® (Verenium) and Axtra® XB (Xylanase/beta-glucanase, DuPont)

In a particular embodiment, the composition of the invention comprises a protease (EC 3.4). Examples of commercially available proteases include Ronozyme® ProAct (DSM Nutritional Products).

### Amino Acids

The composition of the invention may further comprise one or more amino acids. Examples of amino acids which are used in animal feed are lysine, alanine, beta-alanine, threonine, methionine and tryptophan.

### Other Feed Ingredients

The composition of the invention may further comprise colouring agents, stabilisers, growth improving additives and aroma compounds/flavorings, polyunsaturated fatty acids (PUFAs); reactive oxygen generating species, anti-microbial peptides and anti-fungal polypeptides.

Examples of colouring agents are carotenoids such as beta-carotene, astaxanthin, and lutein.

Examples of aroma compounds/flavorings are creosol, anethol, deca-, undeca- and/or dodeca-lactones, ionones, irone, gingerol, piperidine, propylidene phatalide, butylidene phatalide, capsaicin and tannin.

Examples of antimicrobial peptides (AMP's) are CAP18, Leucocin A, Tritrpticin, Protegrin-1, Thanatin, Defensin, Lactoferrin, Lactoferricin, and Ovispirin such as Novispirin (Robert Lehrer, 2000), Plectasins, and Statins, including the compounds and polypeptides disclosed in WO 03/044049 and WO 03/048148, as well as variants or fragments of the above that retain antimicrobial activity.

Examples of antifungal polypeptides (AFP's) are the Aspergillus giganteus, and Aspergillus niger peptides, as well as variants and fragments thereof which retain antifungal activity, as disclosed in WO 94/01459 and WO 02/090384.

Examples of polyunsaturated fatty acids are C18, C20 and C22 polyunsaturated fatty acids, such as arachidonic acid, docosohexaenoic acid, eicosapentaenoic acid and gamma-linoleic acid.

Examples of reactive oxygen generating species are chemicals such as perborate, persulphate, or percarbonate; and enzymes such as an oxidase, an oxygenase or a syntethase.

The composition of the invention may further comprise at least one amino acid. Examples of amino acids which are used in animal feed are lysine, alanine, beta-alanine, threonine, methionine and tryptophan.

### Manufacturing

Animal diets can e.g. be manufactured as mash feed (non-pelleted) or pelleted feed. Typically, the milled feed-stuffs are mixed and sufficient amounts of essential vitamins and minerals are added according to the specifications for the species in question. The bacteria cultures and optionally enzymes can be added as solid or liquid formulations. For example, for mash feed a solid or liquid culture formulation may be added before or during the ingredient mixing step. For pelleted feed the (liquid or solid) culture preparation may also be added before or during the feed ingredient step. Typically a liquid culture preparation comprises the culture of the invention optionally with a polyol, such as glycerol, ethylene glycol or propylene glycol, and is added after the pelleting step, such as by spraying the liquid formulation onto the pellets. The enzyme may also be incorporated in a feed additive or premix.

The enzyme may be added to the feed mix as a granule, which is optionally pelleted or extruded. The granule typically comprises a core particle and one or more coatings, which typically are salt and/or wax coatings. The core particle can either be a homogeneous blend of an active compound optionally together with salts (e.g. organic or inorganic zinc or calcium salt) or an inert particle with an active compound applied onto it. The active compound is the culture of the invention optionally combined with one or more enzymes. The inert particle may be water soluble or water insoluble, e.g. starch, a sugar (such as sucrose or lactose), or a salt (such as NaCl, Na₂SO₄). The salt coating is typically at least 1 μm thick and can either be one particular salt or a mixture of salts, such as Na₂SO₄, K₂SO₄, MgSO₄ and/or sodium citrate. Other examples are those described in e.g. WO 2008/017659, WO 2006/034710, WO 97/05245, WO 98/54980, WO 98/55599, WO 00/70034 or polymer coating such as described in WO 01/00042.

Alternatively, the protease can be prepared by freezing a mixture of liquid culture solution with a bulking agent such as ground soybean meal, and then lyophilizing the mixture.

Methods of Treatment of C. perfringens Infections and/or Necrotic Enteritis

In one embodiment, the invention relates to a method for treatment of C. perfringens infections and/or necrotic enteritis in a mono-gastric animal (such as poultry or swine) using the composition according to the invention. In one embodiment the animal is not a human being. In a further embodiment, the animal feed is fed to a mono-gastric animal. Mono-gastric animals include, but are not limited to, pigs or swine (including, but not limited to, piglets, growing pigs, and sows); poultry such as turkeys, ducks and chicken (including but not limited to broiler chicks, layers); horses (including but not limited to hotbloods, coldbloods and warm bloods) and fish (including but not limited to salmon, trout, tilapia, catfish and carps; and crustaceans (including but not limited to shrimps and prawns). Pigs and/or poultry are preferred mono-gastric animals.

The animal feed can further comprise one or more components selected from the list consisting of concentrate; forage; one or more enzymes; one or more additional microbes; one or more vitamins; one or more minerals; one or more amino acids; and one or more other feed ingredients.

In a further embodiment, the method comprises adding to the animal feed one or more bacterial strains such as at least two of the above strains, at least three of the above strains, at least four of the above strains, at least five of the above strains, at least six of the above strains, up to and including all of the above strains.

In one embodiment, the method comprises adding to the animal feed the Bacillus strains having the deposit accession number NRRL B-50136 (or a strain having all of the identifying characteristics of the strain or a mutant of the strain).

In one embodiment, the method comprises adding to the animal feed the Bacillus strains having the deposit accession number NRRL B-50147 (or a strain having all of the identifying characteristics of the strain or a mutant of the strain).

In one embodiment, the method comprises adding to the animal feed the Bacillus strains having the deposit accession number NRRL B-50141 (or a strain having all of the identifying characteristics of the strain or a mutant of the strain).

In one embodiment, the method comprises adding to the animal feed the Bacillus strains having the deposit accession number PTA-7543 (or a strain having all of the identifying characteristics of the strain or a mutant of the strain).

In one embodiment, the method comprises adding to the animal feed the Bacillus strains having the deposit accession number NRRL B-50888 (or a strain having all of the identifying characteristics of the strain or a mutant of the strain).

In one embodiment, the method comprises adding to the animal feed the Bacillus strains having the deposit accession number PTA-7549 (or a strain having all of the identifying characteristics of the strain or a mutant of the strain).

In one embodiment, the method comprises adding to the animal feed the Bacillus strains having the deposit accession number NRRL B-50349 (or a strain having all of the identifying characteristics of the strain or a mutant of the strain).

In one embodiment, the method comprises adding to the animal feed the Bacillus strains having the deposit accession numbers NRRL B-50136 and NRRL B-50147. In one embodiment, the method comprises adding to the animal feed the Bacillus strains having the deposit accession numbers NRRL B-50136 and NRRL B-50141. In one embodiment, the method comprises adding to the animal feed the Bacillus strains having the deposit accession numbers NRRL B-50136 and PTA-7543. In one embodiment, the method comprises adding to the animal feed the Bacillus strains having the deposit accession numbers NRRL B-50136 and NRRL B-50888. In one embodiment, the method comprises adding to the animal feed the Bacillus strains having the deposit accession numbers NRRL B-50136 and PTA-7549. In one embodiment, the method comprises adding to the animal feed the Bacillus strains having the deposit accession numbers NRRL B-50136 and NRRL B-50349.

In one embodiment, the method comprises adding to the animal feed the Bacillus strains having the deposit accession numbers NRRL B-50147 and NRRL B-50141. In one embodiment, the method comprises adding to the animal feed the Bacillus strains having the deposit accession numbers NRRL B-50147 and PTA-7543. In one embodiment, the method comprises adding to the animal feed the Bacillus strains having the deposit accession numbers NRRL B-50147 and NRRL B-50888. In one embodiment, the method comprises adding to the animal feed the Bacillus strains having the deposit accession numbers NRRL B-50147 and PTA-7549. In one embodiment, the method comprises adding to the animal feed the Bacillus strains having the deposit accession numbers NRRL B-50147 and NRRL B-50349.

In one embodiment, the method comprises adding to the animal feed the Bacillus strains having the deposit accession numbers NRRL B-50141 and PTA-7543. In one embodiment, the method comprises adding to the animal feed the Bacillus strains having the deposit accession numbers NRRL B-50141 and NRRL B-50888. In one embodiment, the method comprises adding to the animal feed the Bacillus strains having the deposit accession numbers NRRL B-50141 and PTA-7549. In one embodiment, the method comprises adding to the animal feed the Bacillus strains having the deposit accession numbers NRRL B-50141 and NRRL B-50349.

In one embodiment, the method comprises adding to the animal feed the Bacillus strains having the deposit accession numbers PTA-7543 and NRRL B-50888. In one embodiment, the method comprises adding to the animal feed the Bacillus strains having the deposit accession numbers PTA-7543 and PTA-7549. In one embodiment, the method comprises adding to the animal feed the Bacillus strains having the deposit accession numbers PTA-7543 and NRRL B-50349.

In one embodiment, the method comprises adding to the animal feed the Bacillus strains having the deposit accession numbers NRRL B-50888 and PTA-7549. In one embodiment, the method comprises adding to the animal feed the Bacillus strains having the deposit accession numbers NRRL B-50888 and NRRL B-50349. In one embodiment, the method comprises adding to the animal feed the Bacillus strains having the deposit accession numbers PTA-7549 and NRRL B-50349.

In a preferred embodiment, the invention relates to a method for treatment of C. perfringens infections in a mono-gastric animal (such as swine or poultry), comprising feeding the mono-gastric animal with an animal feed composition comprising spores of a Bacillus strain wherein:

i. at least 60% (such as at least 65%) of the Bacillus spores survive gastric stability e.g. as described in Example 1; and/or

ii. at least 55% of the Bacillus spores germinate in less than 4 hours e.g. as described in Example 1,

iii. the Bacillus strain has the deposit accession number NRRL B-50136 and

iv. the Bacillus spores have antimicrobial activity against Clostridium perfringens e.g. as described in Example 1.

In a preferred embodiment, the invention relates to a method for treatment of C. perfringens infections in a mono-gastric animal (such as swine or poultry), comprising feeding the mono-gastric animal with an animal feed composition comprising spores of a Bacillus strain wherein:

i. at least 60% (such as at least 65%, 70%, 80% or 90%) of the Bacillus spores survive gastric stability e.g. as described in Example 1; and/or

ii. at least 55% (such as at least 60%, 70%, 80% or 90%) of the Bacillus spores germinate in less than 4 hours e.g. as described in Example 1,

iii. the Bacillus strain has the deposit accession number NRRL B-50147 and

iv. the Bacillus spores have antimicrobial activity against Clostridium perfringens e.g. as described in Example 1.

In a preferred embodiment, the invention relates to a method for treatment of C. perfringens infections in a mono-gastric animal (such as swine or poultry), comprising feeding the mono-gastric animal with an animal feed composition comprising spores of a Bacillus strain wherein:

i. at least 60% (such as at least 65%, 70%, 80% or 90%) of the Bacillus spores survive gastric stability e.g. as described in Example 1; and/or

ii. at least 55% (such as at least 60%, 70%, 80% or 85%) of the Bacillus spores germinate in less than 4 hours e.g. as described in Example 1,

iii. the Bacillus strain has the deposit accession number NRRL B-50141 and

iv. the Bacillus spores have antimicrobial activity against Clostridium perfringens e.g. as described in Example 1.

In a preferred embodiment, the invention relates to a method for treatment of C. perfringens infections in a mono-gastric animal (such as swine or poultry), comprising feeding the mono-gastric animal with an animal feed composition comprising spores of a Bacillus strain wherein:

i. at least 60% (such as at least 65%, 70%, 80% or 90%) of the Bacillus spores survive gastric stability e.g. as described in Example 1; and/or

ii. at least 55% (such as at least 60%, 70%, 80% or 90%) of the Bacillus spores germinate in less than 4 hours e.g. as described in Example 1,

iii. the Bacillus strain has the deposit accession number PTA-7543 and

iv. the Bacillus spores have antimicrobial activity against Clostridium perfringens e.g. as described in Example 1.

In a preferred embodiment, the invention relates to a method for treatment of C. perfringens infections in a mono-gastric animal (such as swine or poultry), comprising feeding the mono-gastric animal with an animal feed composition comprising spores of a

Bacillus strain wherein:

i. at least 60% (such as at least 65%, 70%, or 75%) of the Bacillus spores survive gastric stability e.g. as described in Example 1; and/or

ii. at least 55% (such as at least 60%, 70%, 80% or 90%) of the Bacillus spores germinate in less than 4 hours e.g. as described in Example 1,

iii. the Bacillus strain has the deposit accession number NRRL B-50888 and

iv. the Bacillus spores have antimicrobial activity against Clostridium perfringens e.g. as described in Example 1.

In a preferred embodiment, the invention relates to a method for treatment of C. perfringens infections in a mono-gastric animal (such as swine or poultry), comprising feeding the mono-gastric animal with an animal feed composition comprising spores of a Bacillus strain wherein:

i. at least 60% (such as at least 65%, 70%, 80% or 90%) of the Bacillus spores survive gastric stability e.g. as described in Example 1; and/or

ii. at least 55% (such as at least 60%, 70%, 80% or 85%) of the Bacillus spores germinate in less than 4 hours e.g. as described in Example 1,

iii. the Bacillus strain has the deposit accession number PTA-7549 and

iv. the Bacillus spores have antimicrobial activity against Clostridium perfringens e.g. as described in Example 1.

In a preferred embodiment, the invention relates to a method for treatment of C. perfringens infections in a mono-gastric animal (such as swine or poultry), comprising feeding the mono-gastric animal with an animal feed composition comprising spores of a

Bacillus strain wherein:

i. at least 60% (such as at least 62%) of the Bacillus spores survive gastric stability e.g. as described in Example 1; and/or

ii. at least 55% (such as at least 60%, 70%, 80% or 90%) of the Bacillus spores germinate in less than 4 hours e.g. as described in Example 1,

iii. the Bacillus strain has the deposit accession number NRRL B-50349 and

iv. the Bacillus spores have antimicrobial activity against Clostridium perfringens e.g. as described in Example 1.

In a preferred embodiment, the invention relates to a method for treatment of necrotic enteritis in a mono-gastric animal (such as swine or poultry), comprising feeding the mono-gastric animal with an animal feed composition comprising spores of a Bacillus strain wherein:

i. at least 60% (such as at least 65%) of the Bacillus spores survive gastric stability e.g. as described in Example 1; and/or

ii. at least 55% of the Bacillus spores germinate in less than 4 hours e.g. as described in Example 1,

iii. the Bacillus strain has the deposit accession number NRRL B-50136 and

iv. the Bacillus spores have antimicrobial activity against Clostridium perfringens e.g. as described in Example 1.

In a preferred embodiment, the invention relates to a method for treatment of necrotic enteritis in a mono-gastric animal (such as swine or poultry), comprising feeding the mono-gastric animal with an animal feed composition comprising spores of a Bacillus strain wherein:

i. at least 60% (such as at least 65%, 70%, 80% or 90%) of the Bacillus spores survive gastric stability e.g. as described in Example 1; and/or

ii. at least 55% (such as at least 60%, 70%, 80% or 90%) of the Bacillus spores germinate in less than 4 hours e.g. as described in Example 1,

iii. the Bacillus strain has the deposit accession number NRRL B-50147 and

iv. the Bacillus spores have antimicrobial activity against Clostridium perfringens e.g. as described in Example 1.

In a preferred embodiment, the invention relates to a method for treatment of necrotic enteritis in a mono-gastric animal (such as swine or poultry), comprising feeding the mono-gastric animal with an animal feed composition comprising spores of a Bacillus strain wherein:

i. at least 60% (such as at least 65%, 70%, 80% or 90%) of the Bacillus spores survive gastric stability e.g. as described in Example 1; and/or

ii. at least 55% (such as at least 60%, 70%, 80% or 85%) of the Bacillus spores germinate in less than 4 hours e.g. as described in Example 1,

iii. the Bacillus strain has the deposit accession number NRRL B-50141 and

iv. the Bacillus spores have antimicrobial activity against Clostridium perfringens e.g. as described in Example 1.

In a preferred embodiment, the invention relates to a method for treatment of necrotic enteritis in a mono-gastric animal (such as swine or poultry), comprising feeding the mono-gastric animal with an animal feed composition comprising spores of a Bacillus strain wherein:

i. at least 60% (such as at least 65%, 70%, 80% or 90%) of the Bacillus spores survive gastric stability e.g. as described in Example 1; and/or

ii. at least 55% (such as at least 60%, 70%, 80% or 90%) of the Bacillus spores germinate in less than 4 hours e.g. as described in Example 1,

iii. the Bacillus strain has the deposit accession number PTA-7543 and

iv. the Bacillus spores have antimicrobial activity against Clostridium perfringens e.g. as described in Example 1.

In a preferred embodiment, the invention relates to a method for treatment of necrotic enteritis in a mono-gastric animal (such as swine or poultry), comprising feeding the mono-gastric animal with an animal feed composition comprising spores of a Bacillus strain wherein:

i. at least 60% (such as at least 65%, 70%, or 75%) of the Bacillus spores survive gastric stability e.g. as described in Example 1; and/or

ii. at least 55% (such as at least 60%, 70%, 80% or 90%) of the Bacillus spores germinate in less than 4 hours e.g. as described in Example 1,

iii. the Bacillus strain has the deposit accession number NRRL B-50888 and

iv. the Bacillus spores have antimicrobial activity against Clostridium perfringens e.g. as described in Example 1.

In a preferred embodiment, the invention relates to a method for treatment of necrotic enteritis in a mono-gastric animal (such as swine or poultry), comprising feeding the mono-gastric animal with an animal feed composition comprising spores of a Bacillus strain wherein:

i. at least 60% (such as at least 65%, 70%, 80% or 90%) of the Bacillus spores survive gastric stability e.g. as described in Example 1; and/or

ii. at least 55% (such as at least 60%, 70%, 80% or 85%) of the Bacillus spores germinate in less than 4 hours e.g. as described in Example 1,

iii. the Bacillus strain has the deposit accession number PTA-7549 and

iv. the Bacillus spores have antimicrobial activity against Clostridium perfringens e.g. as described in Example 1.

In a preferred embodiment, the invention relates to a method for treatment of necrotic enteritis in a mono-gastric animal (such as swine or poultry), comprising feeding the mono-gastric animal with an animal feed composition comprising spores of a Bacillus strain wherein:

i. at least 60% (such as at least 62%) of the Bacillus spores survive gastric stability e.g. as described in Example 1; and/or

ii. at least 55% (such as at least 60%, 70%, 80% or 90%) of the Bacillus spores germinate in less than 4 hours e.g. as described in Example 1,

iii. the Bacillus strain has the deposit accession number NRRL B-50349 and

iv. the Bacillus spores have antimicrobial activity against Clostridium perfringens e.g. as described in Example 1.

In another embodiment of the method, the animal feed further comprises concentrate. In another embodiment of the method, the animal feed further comprises forage. In another embodiment of the method, the animal feed further comprises one or more additional microbes. In another embodiment of the method, the animal feed further comprises one or more enzymes. In another embodiment of the method, the animal feed further comprises one or more vitamins. In another embodiment of the method, the animal feed further comprises one or more minerals. In another embodiment of the method, the animal feed further comprises one or more amino acids. In another embodiment of the method, the animal feed further comprises one or more other feed ingredients.

In an embodiment to any of the aforementioned embodiments, the method also improves the health of the mono-gastric animal feed. In another embodiment to any of the aforementioned embodiments, the method also increases the egg yield of poultry. In an embodiment to any of the aforementioned embodiments, the method also increases the meat yield of the mono-gastric animal.

In a preferred embodiment, the method comprises administering to a mono-gastric animal one or more bacterial strains described herein, wherein the bacterial count of each of the bacterial strains is between 1×10⁴ and 1×10¹⁴ CFU/kg of forage, preferably between 1×10⁶ and 1×10¹² CFU/kg of forage, and more preferably between 1×10⁷ and 1×10¹¹ CFU/kg of animal feed. In a more preferred embodiment the bacterial count of each of the bacterial strains described herein is between 1×10⁸ and 1×10¹⁰CFU/kg of animal feed.

In a preferred embodiment, the method comprises administering to a mono-gastric animal one or more bacterial strains described herein, wherein the bacterial count of each of the bacterial strains is between 1×10⁵ and 1×10¹⁵ CFU/animal/day, preferably between 1×10⁷ and 1×10¹³ CFU/animal/day, and more preferably between 1×10⁸ and 1×10¹² CFU/animal/day. In a more preferred embodiment the bacterial count of each of the bacterial strains described herein is between 1×10⁹ and 1×10¹¹CFU/animal/day.

In another aspect, the invention covers the method for treatment of C. perfringens infections comprising:

(a) feeding a mono-gastric animal (such as swine or poultry) a feed; and

(b) administering to an animal one or more Bacillus strains selected from the group consisting of:
the strain having the deposit accession number NRRL B-50136 (or a strain having all of the identifying characteristics of the strain or a mutant of the strain);
the strain having the deposit accession number NRRL B-50147 (or a strain having all of the identifying characteristics of the strain or a mutant of the strain);
the strain having the deposit accession number NRRL B-50141 (or a strain having all of the identifying characteristics of the strain or a mutant of the strain);
the strain having the deposit accession number PTA-7543 (or a strain having all of the identifying characteristics of the strain or a mutant of the strain);
the strain having the deposit accession number NRRL B-50888 (or a strain having all of the identifying characteristics of the strain or a mutant of the strain);
the strain having the deposit accession number PTA-7549 (or a strain having all of the identifying characteristics of the strain or a mutant of the strain); and
the strain having the deposit accession number NRRL B-50349 (or a strain having all of the identifying characteristics of the strain or a mutant of the strain), or any combination thereof,

wherein the Bacillus strain antimicrobial activity against Clostridium perfringens, and

wherein step (a) occurs before, after, or simultaneously with step (b).

In a further embodiment, the method comprises administering to the animal feed one or more bacterial strains such as at least two of the above strains, at least three of the above strains, at least four of the above strains, at least five of the above strains, at least six of the above strains, up to and including all of the above strains.

In a preferred embodiment of the method, the animal feed is fed to a mono-gastric animal. In an embodiment of the method, the mono-gastric animal is e.g. pigs or swine (including, but not limited to, piglets, growing pigs, and sows); poultry such as turkeys, ducks and chicken (including but not limited to broiler chicks, layers); horses (including but not limited to hotbloods, coldbloods and warm bloods), or fish (including but not limited to salmon, trout, tilapia, catfish and carps; and crustaceans (including but not limited to shrimps and prawns).

In another embodiment of the method, the animal feed further comprises one or more components selected from the list consisting of concentrate; forage; one or more enzymes; one or more additional microbes; one or more vitamins; one or more minerals; one or more amino acids; and one or more other feed ingredients.

In another embodiment of the method, the animal feed further comprises concentrate. In another embodiment of the method, the animal feed further comprises forage. In another embodiment of the method, the animal feed further comprises one or more additional microbes. In another embodiment of the method, the animal feed further comprises one or more enzymes. In another embodiment of the method, the animal feed further comprises one or more vitamins. In another embodiment of the method, the animal feed further comprises one or more minerals. In another embodiment of the method, the animal feed further comprises one or more amino acids. In another embodiment of the method, the animal feed further comprises one or more other feed ingredients.

In still yet another embodiment of the method, the one or more bacterial strains are present in the form of a stable spore. In still a further embodiment of the method, the stable spore will germinate in the rumen of the ruminant.

In a preferred embodiment, the method comprises administering to a mono-gastric animal one or more bacterial strains described herein, wherein the bacterial count of each of the bacterial strains is between 1×10⁴ and 1×10¹⁴ CFU/kg of forage, preferably between 1×10⁶ and 1×10¹² CFU/kg of forage, and more preferably between 1×10⁷ and 1×10¹¹ CFU/kg of forage. In a more preferred embodiment the bacterial count of each of the bacterial strains described herein is between 1×10⁸ and 1×10¹⁰CFU/kg of forage.

In a preferred embodiment, the method comprises administering to a mono-gastric animal one or more bacterial strains described herein, wherein the bacterial count of each of the bacterial strains is between 1×10⁵ and 1×10¹⁵ CFU/animal/day, preferably between 1×10⁷ and 1×10¹³ CFU/animal/day, and more preferably between 1×10⁸ and 1×10¹² CFU/animal/day. In a more preferred embodiment the bacterial count of each of the bacterial strains described herein is between 1×10⁹ and 1×10¹¹ CFU/animal/day.

In a preferred embodiment, the method comprises administering to a mono-gastric animal one or more bacterial strains described herein, wherein the bacterial count of each Bacillus spore is between 1×10⁵ and 1×10¹⁵ CFU/animal/day, preferably between 1×10⁷ and 1×10¹³ CFU/animal/day, and more preferably between 1×10⁸ and 1×10¹² CFU/animal/day. The invention relates in a further embodiment to use of the animal feed composition to improve the performance of an animal, such as improving the feed conversion ratio, improving the body weight gain and/or improving the feed efficiency and/or improving the health.

### PREFERRED EMBODIMENTS

Preferred embodiments of the invention are described in the sets of items herein below.

### Item Set 1:

1. An animal feed composition comprising spores of a Bacillus strain wherein:

i. the Bacillus strain is a Bacillus subtilis strain or a Bacillus amyloliquefaciens strain;

ii. at least 60% of the Bacillus spores survive gastric stability;

iii. at least 55% of the Bacillus spores germinate in less than 4 hours,

iv. the Bacillus spores have antimicrobial activity against Clostridium perfringens and

v. the Bacillus strain has enzyme activity under aerobic conditions against one or more of the substrates selected from the group consisting of Amylose, Arabinan, Arabinoxylan, Casein and Xylan.

2. An animal feed composition comprising spores of a Bacillus strain wherein:

i. the Bacillus strain is a Bacillus subtilis strain or a Bacillus amyloliquefaciens strain;

ii. at least 60% of the Bacillus spores survive gastric stability;

iii. at least 55% of the Bacillus spores germinate in less than 4 hours,

iv. the Bacillus spores have antimicrobial activity against Clostridium perfringens and

v. the Bacillus strain has enzyme activity under anaerobic conditions against one or more of the substrates selected from the group consisting of Amylose, Arabinan, Arabinoxylan, Casein and Xylan.

3. An animal feed composition comprising spores of a Bacillus strain wherein:

i. at least 60% of the Bacillus spores survive gastric stability;

ii. at least 55% of the Bacillus spores germinate in less than 4 hours,

iii. the Bacillus strain is selected from the group consisting of:
the strain having the deposit accession number NRRL B-50136;
the strain having the deposit accession number NRRL B-50147;
the strain having the deposit accession number NRRL B-50141;
the strain having the deposit accession number PTA-7543;
the strain having the deposit accession number NRRL B-50888;
the strain having the deposit accession number PTA-7549; and
the strain having the deposit accession number NRRL B-50349, or any combination thereof, and

iv. the Bacillus spores have antimicrobial activity against Clostridium perfringens.

4. The animal feed composition of item 1 to 3 wherein the Bacillus spore kills/inhibits 10% (such as at least 20%, at least 30%, at least 40%, at least 50%, at least 60%, at least 70%, at least 80% or at least 90%) of Clostridium perfringens after 24 hours.
5. The animal feed composition of any of items 1 to 4 wherein at least 70% (such as at least 80% or at least 90%) of the Bacillus spores survive gastric stability.
6. The animal feed composition of any of items 1 to 5 wherein at least 80% (such as at least 85%, at least 90% or at least 95%) of the Bacillus spores germinate in less than 4 hours.
7. The animal feed composition of any of items 1 to 6 which further comprises a formulating agent.
8. The animal feed composition of item 7 wherein the formulating agent comprises one or more of the following compounds: glycerol, ethylene glycol, 1, 2-propylene glycol, 1, 3-propylene glycol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch and cellulose.
9. The animal feed composition of any of items 1 to 8 which further comprises one or more components selected from the list consisting of:

one or more enzymes;

one or more additional microbes;

one or more vitamins;

one or more minerals;

one or more amino acids; and

one or more other feed ingredients.

10. The animal feed composition of any of items 1 to 9 wherein the bacterial count of each Bacillus spore is between 1×10⁵ and 1×10¹⁵ CFU/animal/day, preferably between 1×10⁷ and 1×10¹³ CFU/animal/day, and more preferably between 1×10⁸ and 1×10¹² CFU/animal/day.
11. A mono-gastric animal feed comprising the animal feed composition of any of items 1 to 10.
12. The mono-gastric animal feed of item 11 wherein the mono-gastric animal is selected from the list consisting of: pigs, swine, piglets, sows, poultry, turkeys, ducks, chicken, broilers, layers, chicks, fish and crustaceans.
13. A composition comprising the animal feed composition of any of items 1 to 12 for the treatment of necrotic enteritis or the treatment of a Clostridium perfringens infection.
14. The composition of item 13 for the treatment of mono-gastric animals.
15. A method of treating a Clostridium perfringens infection or for treating necrotic enteritis in an animal comprising administrating the animal feed composition of any of items 1 to 12.
16. Use of the animal feed composition of any of items 1 to 12 to improve the performance of an animal, such as improving the feed conversion ratio, improving the body weight gain and/or improving the feed efficiency and/or improving the health.

### Item Set 2:

1. A Bacillus subtilis strain or a Bacillus amyloliquefaciens strain characterized in that

i. at least 60% of the Bacillus spores survive gastric stability;

ii. at least 55% of the Bacillus spores germinate in less than 4 hours,

iii. the Bacillus spores have antimicrobial activity against Clostridium perfringens and

iv. the Bacillus strain has enzyme activity under aerobic conditions against one or more of the substrates selected from the group consisting of Amylose, Arabinan, Arabinoxylan, Casein and Xylan

for treatment of a Clostridium perfringens infection.
2. The Bacillus strain according to item 1, wherein the Bacillus strain is selected from the group consisting of

the strain having the deposit accession number NRRL B-50136 or a strain having all of the identifying characteristics of Bacillus NRRL B-50136 or a mutant thereof;

the strain having the deposit accession number NRRL B-50147 or a strain having all of the identifying characteristics of Bacillus NRRL B-50147 or a mutant thereof;

the strain having the deposit accession number NRRL B-50141 or a strain having all of the identifying characteristics of Bacillus NRRL B-50141 or a mutant thereof;

the strain having the deposit accession number PTA-7543 or a strain having all of the identifying characteristics of Bacillus PTA-7543 or a mutant thereof;

the strain having the deposit accession number NRRL B-50888 or a strain having all of the identifying characteristics of Bacillus NRRL B-50888 or a mutant thereof;

the strain having the deposit accession number PTA-7549 or a strain having all of the identifying characteristics of Bacillus PTA-7549 or a mutant thereof;

the strain having the deposit accession number B-50349 or a strain having all of the identifying characteristics of Bacillus B-50349 or a mutant thereof; or any combination thereof.

3. A Bacillus strain selected from the group consisting of

the strain having the deposit accession number NRRL B-50136 or a strain having all of the identifying characteristics of Bacillus NRRL B-50136 or a mutant thereof;

the strain having the deposit accession number NRRL B-50147 or a strain having all of the identifying characteristics of Bacillus NRRL B-50147 or a mutant thereof;

the strain having the deposit accession number NRRL B-50141 or a strain having all of the identifying characteristics of Bacillus NRRL B-50141 or a mutant thereof;

the strain having the deposit accession number PTA-7543 or a strain having all of the identifying characteristics of Bacillus PTA-7543 or a mutant thereof;

the strain having the deposit accession number NRRL B-50888 or a strain having all of the identifying characteristics of Bacillus NRRL B-50888 or a mutant thereof;

the strain having the deposit accession number PTA-7549 or a strain having all of the identifying characteristics of Bacillus PTA-7549 or a mutant thereof;

the strain having the deposit accession number B-50349 or a strain having all of the identifying characteristics of Bacillus B-50349 or a mutant thereof; or any combination thereof for treatment of a Clostridium perfringens infection.

4. The Bacillus strain according to any of items 1 to 3, wherein the treatment is treatment of a Clostridium perfringens infection in poultry.
5. The Bacillus strain according to any of items 1 to 3, wherein the treatment is treatment of a Clostridium perfringens infection in swine.
6. The Bacillus strain according to any of items 1 to 3, wherein the treatment comprises administration of an animal feed comprising the Bacillus strain to poultry or swine.
7. A method for improving the performance of poultry or swine comprising feeding the poultry or swine with a Bacillus subtilis strain or a Bacillus amyloliquefaciens strain characterized in that

i. at least 60% of the Bacillus spores survive gastric stability;

ii. at least 55% of the Bacillus spores germinate in less than 4 hours,

iii. the Bacillus spores have antimicrobial activity against Clostridium perfringens and

iv. the Bacillus strain has enzyme activity under aerobic conditions against one or more of the substrates selected from the group consisting of Amylose, Arabinan, Arabinoxylan, Casein and Xylan.

8. The method according to item 7, wherein the Bacillus strain is selected from the group consisting of

the strain having the deposit accession number NRRL B-50136 or a strain having all of the identifying characteristics of Bacillus NRRL B-50136 or a mutant thereof;

the strain having the deposit accession number NRRL B-50147 or a strain having all of the identifying characteristics of Bacillus NRRL B-50147 or a mutant thereof;

the strain having the deposit accession number NRRL B-50141 or a strain having all of the identifying characteristics of Bacillus NRRL B-50141 or a mutant thereof;

the strain having the deposit accession number PTA-7543 or a strain having all of the identifying characteristics of Bacillus PTA-7543 or a mutant thereof;

the strain having the deposit accession number NRRL B-50888 or a strain having all of the identifying characteristics of Bacillus NRRL B-50888 or a mutant thereof;

the strain having the deposit accession number PTA-7549 or a strain having all of the identifying characteristics of Bacillus PTA-7549 or a mutant thereof;

the strain having the deposit accession number B-50349 or a strain having all of the identifying characteristics of Bacillus B-50349 or a mutant thereof; or any combination thereof.

9. A method for improving the performance of poultry or swine comprising feeding the poultry or swine with a Bacillus strain selected from the group consisting of

the strain having the deposit accession number NRRL B-50136 or a strain having all of the identifying characteristics of Bacillus NRRL B-50136 or a mutant thereof;

the strain having the deposit accession number NRRL B-50147 or a strain having all of the identifying characteristics of Bacillus NRRL B-50147 or a mutant thereof;

the strain having the deposit accession number NRRL B-50141 or a strain having all of the identifying characteristics of Bacillus NRRL B-50141 or a mutant thereof;

the strain having the deposit accession number PTA-7543 or a strain having all of the identifying characteristics of Bacillus PTA-7543 or a mutant thereof;

the strain having the deposit accession number NRRL B-50888 or a strain having all of the identifying characteristics of Bacillus NRRL B-50888 or a mutant thereof;

the strain having the deposit accession number PTA-7549 or a strain having all of the identifying characteristics of Bacillus PTA-7549 or a mutant thereof;

the strain having the deposit accession number B-50349 or a strain having all of the identifying characteristics of Bacillus B-50349 or a mutant thereof; or any combination thereof.

10. The method according to any of items 7 to 9, wherein the improved performance comprises one or more improvements selected from the group consisting of increased body weight gain, improved feed conversion rate, improving the feed efficiency, improving health and decreased mortality.
11. A poultry or swine feed comprising a Bacillus subtilis strain or a Bacillus amyloliquefaciens strain characterized in that

i. at least 60% of the Bacillus spores survive gastric stability;

ii. at least 55% of the Bacillus spores germinate in less than 4 hours,

iii. the Bacillus spores have antimicrobial activity against Clostridium perfringens and

iv. the Bacillus strain has enzyme activity under aerobic conditions against one or more of the substrates selected from the group consisting of Amylose, Arabinan, Arabinoxylan, Casein and Xylan.

12. The poultry or swine feed according to item 11, wherein the Bacillus strain is selected from the group consisting of

the strain having the deposit accession number NRRL B-50136 or a strain having all of the identifying characteristics of Bacillus NRRL B-50136 or a mutant thereof;

the strain having the deposit accession number NRRL B-50147 or a strain having all of the identifying characteristics of Bacillus NRRL B-50147 or a mutant thereof;

the strain having the deposit accession number NRRL B-50141 or a strain having all of the identifying characteristics of Bacillus NRRL B-50141 or a mutant thereof;

the strain having the deposit accession number PTA-7543 or a strain having all of the identifying characteristics of Bacillus PTA-7543 or a mutant thereof;

the strain having the deposit accession number NRRL B-50888 or a strain having all of the identifying characteristics of Bacillus NRRL B-50888 or a mutant thereof;

the strain having the deposit accession number PTA-7549 or a strain having all of the identifying characteristics of Bacillus PTA-7549 or a mutant thereof;

the strain having the deposit accession number B-50349 or a strain having all of the identifying characteristics of Bacillus B-50349 or a mutant thereof; or any combination thereof.

13. A poultry or swine feed comprising a Bacillus subtilis strain or a Bacillus amyloliquefaciens strain selected from the group consisting of

the strain having the deposit accession number NRRL B-50136 or a strain having all of the identifying characteristics of Bacillus NRRL B-50136 or a mutant thereof;

the strain having the deposit accession number NRRL B-50147 or a strain having all of the identifying characteristics of Bacillus NRRL B-50147 or a mutant thereof;

the strain having the deposit accession number NRRL B-50141 or a strain having all of the identifying characteristics of Bacillus NRRL B-50141 or a mutant thereof;

the strain having the deposit accession number PTA-7543 or a strain having all of the identifying characteristics of Bacillus PTA-7543 or a mutant thereof;

the strain having the deposit accession number NRRL B-50888 or a strain having all of the identifying characteristics of Bacillus NRRL B-50888 or a mutant thereof;

the strain having the deposit accession number PTA-7549 or a strain having all of the identifying characteristics of Bacillus PTA-7549 or a mutant thereof;

the strain having the deposit accession number B-50349 or a strain having all of the identifying characteristics of Bacillus B-50349 or a mutant thereof; or any combination thereof.

14. The Bacillus strain according to any of items 1 to 6, the method according to any of items 7 to 10 or the feed composition according to any of items 11 to 13, wherein the Bacillus is in spore form and kills/inhibits 10% (such as at least 20%, at least 30%, at least 40%, at least 50%, at least 60%, at least 70%, at least 80% or at least 90%) of Clostridium perfringens after 24 hours.
15. The Bacillus strain according to any of items 1 to 6, the method according to any of items 7 to 10 or the feed composition according to any of items 11 to 13, wherein the Bacillus is in spore form and at least 70% (such as at least 80% or at least 90%) of the Bacillus spores survive gastric stability.
16. The Bacillus strain according to any of items 1 to 6, the method according to any of items 7 to 10 or the feed composition according to any of items 11 to 13, wherein the Bacillus is in spore form and at least 80% (such as at least 85%, at least 90% or at least 95%) of the Bacillus spores germinate in less than 4 hours.
17. The animal feed composition of any of items 11 to 13 which further comprises a formulating agent.
18. The animal feed composition of item 17 wherein the formulating agent comprises one or more of the following compounds: glycerol, ethylene glycol, 1, 2-propylene glycol, 1, 3-propylene glycol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch and cellulose.
19. The animal feed composition of item 17 or 18 which further comprises one or more components selected from the list consisting of:

one or more enzymes;

one or more additional microbes;

one or more vitamins;

one or more minerals;

one or more amino acids; and

one or more other feed ingredients.

20. The Bacillus strain according to any of items 1 to 6, wherein the treatment comprises administration of a bacterial count of each Bacillus (e.g. in spore form) between 1×10⁵ and 1×10¹⁵ CFU/animal/day, preferably between 1×10⁷ and 1×10¹³ CFU/animal/day, and more preferably between 1×10⁸ and 1×10¹² CFU/animal/day.
21. The method according to any of items 7 to 10, wherein the method comprises administration of a bacterial count of each Bacillus (e.g. in spore form) between 1×10⁵ and 1×10¹⁵ CFU/animal/day, preferably between 1×10⁷ and 1×10¹³ CFU/animal/day, and more preferably between 1×10⁸ and 1×10¹² CFU/animal/day.
22. The Bacillus strain according to any of items 1 to 6, the method according to any of items 7 to 10 or the feed composition according to any of items 11 to 13 wherein the Bacillus is in spore form.

### Item Set 3:

1. A method of treating a Clostridium perfringens infection comprising administering a Bacillus subtilis strain or a Bacillus amyloliquefaciens strain to poultry or swine wherein the Bacillus strain is characterized in that

i. at least 60% of the Bacillus spores survive gastric stability;

ii. at least 55% of the Bacillus spores germinate in less than 4 hours,

iii. the Bacillus spores have antimicrobial activity against Clostridium perfringens and

iv. the Bacillus strain has enzyme activity under aerobic conditions against one or more of the substrates selected from the group consisting of Amylose, Arabinan, Arabinoxylan, Casein and Xylan.

2. The method according to item 1, wherein the Bacillus strain is selected from the group consisting of

the strain having the deposit accession number NRRL B-50136 or a strain having all of the identifying characteristics of Bacillus NRRL B-50136 or a mutant thereof;

the strain having the deposit accession number NRRL B-50147 or a strain having all of the identifying characteristics of Bacillus NRRL B-50147 or a mutant thereof;

the strain having the deposit accession number NRRL B-50141 or a strain having all of the identifying characteristics of Bacillus NRRL B-50141 or a mutant thereof;

the strain having the deposit accession number PTA-7543 or a strain having all of the identifying characteristics of Bacillus PTA-7543 or a mutant thereof;

the strain having the deposit accession number NRRL B-50888 or a strain having all of the identifying characteristics of Bacillus NRRL B-50888 or a mutant thereof;

the strain having the deposit accession number PTA-7549 or a strain having all of the identifying characteristics of Bacillus PTA-7549 or a mutant thereof;

the strain having the deposit accession number B-50349 or a strain having all of the identifying characteristics of Bacillus B-50349 or a mutant thereof; or any combination thereof.

3. A method of treating a Clostridium perfringens infection comprising administering a Bacillus subtilis strain or a Bacillus amyloliquefaciens strain to poultry or swine wherein the Bacillus strain is selected from the group consisting of

the strain having the deposit accession number NRRL B-50136 or a strain having all of the identifying characteristics of Bacillus NRRL B-50136 or a mutant thereof;

the strain having the deposit accession number NRRL B-50147 or a strain having all of the identifying characteristics of Bacillus NRRL B-50147 or a mutant thereof;

the strain having the deposit accession number NRRL B-50141 or a strain having all of the identifying characteristics of Bacillus NRRL B-50141 or a mutant thereof;

the strain having the deposit accession number PTA-7543 or a strain having all of the identifying characteristics of Bacillus PTA-7543 or a mutant thereof;

the strain having the deposit accession number NRRL B-50888 or a strain having all of the identifying characteristics of Bacillus NRRL B-50888 or a mutant thereof;

the strain having the deposit accession number PTA-7549 or a strain having all of the identifying characteristics of Bacillus PTA-7549 or a mutant thereof;

the strain having the deposit accession number B-50349 or a strain having all of the identifying characteristics of Bacillus B-50349 or a mutant thereof; or any combination thereof.

4. The method according to any of items 1 to 3, wherein the method comprises administration of an animal feed comprising the Bacillus strain to poultry or swine.
5. A method for improving the performance of poultry or swine comprising feeding the poultry or swine with a Bacillus subtilis strain or a Bacillus amyloliquefaciens strain characterized in that

i. at least 60% of the Bacillus spores survive gastric stability;

ii. at least 55% of the Bacillus spores germinate in less than 4 hours,

iii. the Bacillus spores have antimicrobial activity against Clostridium perfringens and

iv. the Bacillus strain has enzyme activity under aerobic conditions against one or more of the substrates selected from the group consisting of Amylose, Arabinan, Arabinoxylan, Casein and Xylan.

6. The method according to item 5, wherein the Bacillus strain is selected from the group consisting of

the strain having the deposit accession number NRRL B-50136 or a strain having all of the identifying characteristics of Bacillus NRRL B-50136 or a mutant thereof;

the strain having the deposit accession number NRRL B-50147 or a strain having all of the identifying characteristics of Bacillus NRRL B-50147 or a mutant thereof;

the strain having the deposit accession number NRRL B-50141 or a strain having all of the identifying characteristics of Bacillus NRRL B-50141 or a mutant thereof;

the strain having the deposit accession number PTA-7543 or a strain having all of the identifying characteristics of Bacillus PTA-7543 or a mutant thereof;

the strain having the deposit accession number NRRL B-50888 or a strain having all of the identifying characteristics of Bacillus NRRL B-50888 or a mutant thereof;

the strain having the deposit accession number PTA-7549 or a strain having all of the identifying characteristics of Bacillus PTA-7549 or a mutant thereof;

the strain having the deposit accession number B-50349 or a strain having all of the identifying characteristics of Bacillus B-50349 or a mutant thereof; or any combination thereof.

7. A method for improving the performance of poultry or swine comprising feeding the poultry or swine with a Bacillus subtilis strain selected from the group consisting of

the strain having the deposit accession number NRRL B-50136 or a strain having all of the identifying characteristics of Bacillus NRRL B-50136 or a mutant thereof;

the strain having the deposit accession number NRRL B-50147 or a strain having all of the identifying characteristics of Bacillus NRRL B-50147 or a mutant thereof;

the strain having the deposit accession number NRRL B-50141 or a strain having all of the identifying characteristics of Bacillus NRRL B-50141 or a mutant thereof;

the strain having the deposit accession number PTA-7543 or a strain having all of the identifying characteristics of Bacillus PTA-7543 or a mutant thereof;

the strain having the deposit accession number NRRL B-50888 or a strain having all of the identifying characteristics of Bacillus NRRL B-50888 or a mutant thereof;

the strain having the deposit accession number PTA-7549 or a strain having all of the identifying characteristics of Bacillus PTA-7549 or a mutant thereof;

the strain having the deposit accession number B-50349 or a strain having all of the identifying characteristics of Bacillus B-50349 or a mutant thereof; or any combination thereof.

8. The method according to any of items 5 to 7, wherein the improved performance comprises one or more improvements selected from the group consisting of increased body weight gain, improved feed conversion rate, improving the feed efficiency, improving health and decreased mortality.
9. A poultry or swine feed comprising a Bacillus subtilis strain or a Bacillus amyloliquefaciens strain characterized in that

i. at least 60% of the Bacillus spores survive gastric stability;

ii. at least 55% of the Bacillus spores germinate in less than 4 hours,

iii. the Bacillus spores have antimicrobial activity against Clostridium perfringens and

iv. the Bacillus strain has enzyme activity under aerobic conditions against one or more of the substrates selected from the group consisting of Amylose, Arabinan, Arabinoxylan, Casein and Xylan.

10. The poultry or swine feed according to item 9, wherein the Bacillus strain is selected from the group consisting of

the strain having the deposit accession number NRRL B-50136 or a strain having all of the identifying characteristics of Bacillus NRRL B-50136 or a mutant thereof;

the strain having the deposit accession number NRRL B-50147 or a strain having all of the identifying characteristics of Bacillus NRRL B-50147 or a mutant thereof;

the strain having the deposit accession number NRRL B-50141 or a strain having all of the identifying characteristics of Bacillus NRRL B-50141 or a mutant thereof;

the strain having the deposit accession number PTA-7543 or a strain having all of the identifying characteristics of Bacillus PTA-7543 or a mutant thereof;

the strain having the deposit accession number NRRL B-50888 or a strain having all of the identifying characteristics of Bacillus NRRL B-50888 or a mutant thereof;

the strain having the deposit accession number PTA-7549 or a strain having all of the identifying characteristics of Bacillus PTA-7549 or a mutant thereof;

the strain having the deposit accession number B-50349 or a strain having all of the identifying characteristics of Bacillus B-50349 or a mutant thereof; or any combination thereof.

11. A poultry or swine feed comprising a Bacillus subtilis strain or a Bacillus amyloliquefaciens strain selected from the group consisting of

the strain having the deposit accession number NRRL B-50136 or a strain having all of the identifying characteristics of Bacillus NRRL B-50136 or a mutant thereof;

the strain having the deposit accession number NRRL B-50147 or a strain having all of the identifying characteristics of Bacillus NRRL B-50147 or a mutant thereof;

the strain having the deposit accession number NRRL B-50141 or a strain having all of the identifying characteristics of Bacillus NRRL B-50141 or a mutant thereof;

the strain having the deposit accession number PTA-7543 or a strain having all of the identifying characteristics of Bacillus PTA-7543 or a mutant thereof;

the strain having the deposit accession number NRRL B-50888 or a strain having all of the identifying characteristics of Bacillus NRRL B-50888 or a mutant thereof;

the strain having the deposit accession number PTA-7549 or a strain having all of the identifying characteristics of Bacillus PTA-7549 or a mutant thereof;

the strain having the deposit accession number B-50349 or a strain having all of the identifying characteristics of Bacillus B-50349 or a mutant thereof; or any combination thereof for treatment of a Clostridium perfringens infection.

12. The method according to any of items 1 to 8 or the feed composition according to any of items 9 to 11, wherein the Bacillus is in spore form and kills/inhibits 10% (such as at least 20%, at least 30%, at least 40%, at least 50%, at least 60%, at least 70%, at least 80% or at least 90%) of Clostridium perfringens after 24 hours.
13. The method according to any of items 1 to 8 or the feed composition according to any of items 9 to 11, wherein the Bacillus is in spore form and at least 70% (such as at least 80% or at least 90%) of the Bacillus spores survive gastric stability.
14. The method according to any of items 1 to 8 or the feed composition according to any of items 9 to 11, wherein the Bacillus is in spore form and at least 80% (such as at least 85%, at least 90% or at least 95%) of the Bacillus spores germinate in less than 4 hours.
15. The animal feed composition of any of items 9 to 11 which further comprises a formulating agent.
16. The animal feed composition of item 15 wherein the formulating agent comprises one or more of the following compounds: glycerol, ethylene glycol, 1, 2-propylene glycol, 1, 3-propylene glycol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch and cellulose.
17. The animal feed composition of any of items 9 to 11 which further comprises one or more components selected from the list consisting of:

one or more enzymes;

one or more additional microbes;

one or more vitamins;

one or more minerals;

one or more amino acids; and

one or more other feed ingredients.

18. The method according to any of items 1 to 8 or the feed composition according to any of items 9 to 11 wherein the bacterial count of each Bacillus (e.g. in spore form) is between
1×10⁵ and 1×10¹⁵ CFU/animal/day, preferably between 1×10⁷ and 1×10¹³ CFU/animal/day, and more preferably between 1×10⁸ and 1×10¹² CFU/animal/day.
19. The method according to any of items 1 to 8 or the feed composition according to any of items 9 to 11 wherein the Bacillus is in spore form.

### EXAMPLES

### Example 1

### Pelleting/Heat Stability at 90° C.

Spores were incorporated into mash and pelleted at 90° C. using pelleting machinery. Samples of the pre-pelleted material (mash) and the pelleted feed were analyzed by plate counting to determine recovery of spores.

Plate counting was performed as follows:

1) Grind pelleted feed or mash in a coffee grinder for about 15 seconds.

2) Weigh 1 gram of material into 99 ml sterile phosphate buffer.

3) Shake for 30 minutes.

4) Remove 1 ml material and place in eppendorf tube. Heat for 20 minutes at 80° C.

5) Serially dilute in sterile phosphate buffer.

6) Plate in triplicate on Standard Methods Agar and grow overnight at 35° C.

7) Count plates to determine level in feed.

Data shown below is an average of 3 replicate bags per strain for both the mash and the pellets. Spore survival and log loss from pelleting were both determined by the difference between spore counts in the mash compared to the pellet. Competitor products (Bacillus subtilis PB6, Clostat; Bacillus subtilis DSM 17231, Gallipro; and Bacillus subtilis DSM 17229, GalliPro Max) were also included for comparison.

Table 1: Summary of Spore Pelleting Stability (Heat Survival) when Pelleted at 90° C.

TABLE 1
Summary of spore pelleting stability (heat survival)
when pelleted at 90° C.

| | SPORE | SPORE | Mash | Pellet |
|---|---|---|---|---|
| | Survival | Log Loss | CFU/kg | CFU/kg |
| NRRL B-50136 | 98.00% | 0.01 | 6.50E+08 | 6.37E+08 |
| NRRL B-50147 | 108.80% | −0.04 | 3.77E+08 | 4.10E+08 |
| NRRL B-50141 | 46.90% | 0.33 | 2.02E+09 | 9.47E+08 |
| PTA-7543 | 38.20% | 0.42 | 1.29E+09 | 4.93E+08 |
| NRRL B-50888 | 35.40% | 0.45 | 2.48E+09 | 8.77E+08 |
| PTA-7549 | 71.30% | 0.15 | 1.09E+09 | 7.77E+08 |
| NRRL B-50349 | 175.30% | −0.24 | 7.30E+08 | 1.28E+09 |
| Bacillus subtilis | 91.60% | 0.04 | 8.00E+07 | 7.33E+07 |
| PB6, Clostat | | | | |
| Bacillus subtilis | 88.00% | 0.06 | 8.10E+08 | 7.13E+08 |
| DSM 17231, | | | | |
| Gallipro | | | | |
| Bacillus subtilis | 52.80% | 0.28 | 2.90E+08 | 1.53E+08 |
| DSM 17229, | | | | |
| GalliPro Max | | | | |

Hemolysis

Hemolysis screening was done according to EFSA recommended guidelines in effect during 2013 and early 2014. These required using 5% sheep's blood agar, incubation at

30° C. for 48 hours, and a specific positive and negative control strain for comparison.
Positive control: Bacillus subtilis strain ATCC21332.
Negative control: Bacillus subtilis type strain NN018422.
Based on the above the strains were classified as being positive or negative with respect to hemolysis (cf. table 2 herein below).
Anti-Clostridium perfringens (Anti-CP) Screening

Anti-CP screening was determined using 3 different tests, cross-streaking, and well diffusion either with culture (whole cell), or with cell-free supernatant.

### Cross-Streaking Assay:

Bacillus isolates were tested to see if they could inhibit Clostridium perfringens ATCC13124. Following overnight growth in 10 ml Tryptic Soy broth with 0.6% yeast extract at 35° C. with continual shaking, culture was applied to Tryptic soy agar with yeast extract at pH 6.2. A single streak of Clostridium perfringens was added to the plate, followed by a perpendicular streak of a Bacillus culture. Plates were incubated in Oxoid jars using anaerogen oxygen-absorbing packets and incubated overnight at 35° C. As the strains grow together, Bacillus able to inhibit Clostridium perfringens growth will show a disruption in the Clostridium perfringens streak where the two strains bisect.

### Well Diffusion Assay:

Well Diffusion Assays were performed to test both whole cells (from an overnight culture) and cell-free supernatant. In these tests, Tryptic Soy agar supplemented with 0.6% yeast extract at pH 6.2 was inoculated 1:1000 (500 uL per 500 ml agar) with an overnight culture of C. perfringens ATCC13124, and plated. After the agar hardened, 4 8 mm diameter wells were punched out of the agar. Each plate contained three replicate wells with 75 ul of culture (whole cell) or cell-free supernatant. The fourth well was a negative control of either sterile tryptic soy broth or 0.22 micron filtered supernatant from sterile broth. After overnight incubation in anaerobic conditions (oxoid jars with anaerogen packs), at 35° C., the plates were examined. Zones of clearing around the wells indicated anti-Clostridium ability by the Bacillus in the wells.

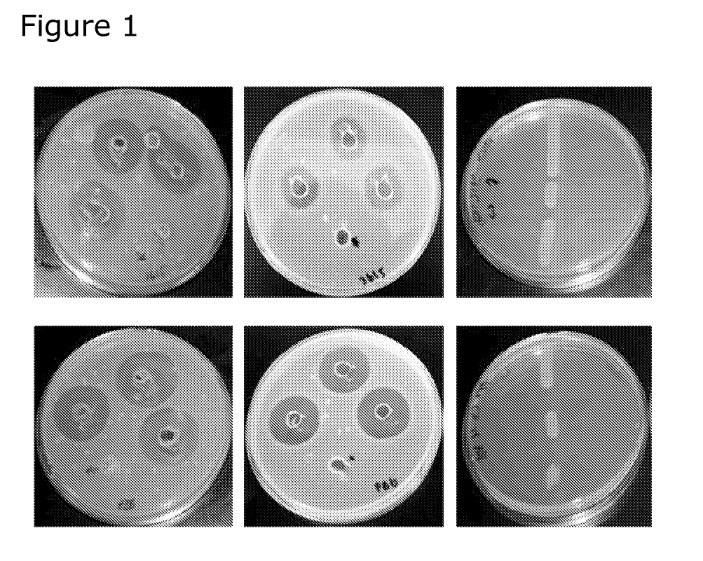

FIG. 1 shows In vitro Screening of Anti-Clostridium perfringens Activity in NRRL B 50349 and ATCC PTA-6737 (PB6). FIG. 1 Top row shows the result with respect to NRRL B-50349. Whole cell well diffusion (left), Supernatant well diffusion (center), Antagonism/Cross Streak (right). Bottom row in FIG. 1 shows the results with respect to ATCC PTA-6737 (PB6) (Competitor strain Clostat). Whole cell well diffusion (left), Supernatant well diffusion (center), Antagonism/Cross Streak (right). In well diffusion assay wells in the bottom center, marked with *, are negative controls. In cross-streak assays, Bacillus is streaked horizontally, Clostridium perfringens vertically.

TABLE 2
Summary of Bacillus anti-CP ability and hemolysis.

| | | | | Anti-CP | Anti-CP | |
|---|---|---|---|---|---|---|
| | | | Anti-CP | Well- | Well- | |
| | | | Cross | Diffusion; | Diffusion; | |
| Strain ID | Genus | Species | Streak | Whole Cell | Supernatant | Hemolysis |
| NRRL | Bacillus | subtilis | + | + | + | + |
| B-50136 | | | | | | |
| NRRL | Bacillus | amyloliquefaciens | + | − | − | + |
| B-50147 | | | | | | |
| NRRL | Bacillus | amyloliquefaciens | − | + | − | + |
| B-50141 | | | | | | |
| ATCC PTA- | Bacillus | amyloliquefaciens | + | + | + | + |
| 7543 | | | | | | |
| NRRL | Bacillus | amyloliquefaciens | + | + | + | + |
| B-50888 | | | | | | |
| ATCC PTA- | Bacillus | amyloliquefaciens | + | + | + | + |
| 7549 | | | | | | |
| NRRL | Bacillus | amyloliquefaciens | + | + | + | + |
| B-50349 | | | | | | |

In Table 2 + indicates that the strain has anti-Clostridium perfringens ability, or that it is hemolytic positive.

In Table 2 − indicates that the strain does not have anti-Clostridium perfringens ability, or that it is hemolytic negative.

### Gastric Survival & Germination

The Bacillus strains were tested for ability to germinate under in vitro conditions that are simulating the conditions of the intestinal system in chicken. The benchmark strains were Bacillus subtilis (isolated from Kemin product, clostat); DSM 17229, Chr Hansen and Gallipro Tect, isolated from Chr Hansen product.

Spore suspensions were diluted in PBS (PBS is Na₂HPO₄.2H₂O 8.77 g/L, KH₂PO₄ 3 g/L, NaCl 4 g/L and MgSO₄.7H₂O 0.2 g/L sterilized by autoclaving) to final concentration of 10⁹ cfu/m L.

A cfu/determination of the spore suspension was made by 100× dilution in PBS followed by pasteurization by incubation at 80° C. for 30 min. Two 100× dilutions were made and 100 μl were spread on TY agar plates. Plates were incubated O/N at 30° C. and colonies were counted. Average plate count=cfu₀.

The spore suspension was diluted to 10⁷ cfu/mL in BA salt pH 3 (BA salt is (NH₄)₂SO₄) 1 g/L, MgSO₄.7H₂O 2.5 g/L, CaCl₂.2H₂O 1.25 g/L, KH₂PO₄ 15 g/L, sterilized by autoclaving), and incubated at 37° C. for 30 min.

A cfu/determination was made by making two 100× dilutions and spreading 100 μl on TY agar plates. Plates were incubated 0/N at 30° C. colonies were counted. Average plate count=cfu_{Gast}.

The spore suspension in BA salt was further diluted to 10⁵ cfu/mL by taking 15 μl spore suspension into a tube containing 1.5 ml MRS-PB broth (MRS-PB broth is MRS broth (Difco art. 288130) added Pancreatin from porcine pancreas (Sigma art P3292) 1.3 mg/mL and Bile extract porcine (Sigma art B8631) 0.6 mg/mL. The medium was sterile filtered after solubilization of pancreatin and bile salt. The tube was incubated at 37° C. 300 rpm agitation for 4 hours. Pasteurize by incubation at 80° C. for 30 min.

A cfu/determination was made by making a 100× dilutions and spreading 100 μl on TY agar plates. Plates were incubated 0/N at 30° C. and colonies were counted. Average plate count=cfu_{Bile}.

### Calculations:

Gastric survival was calculated as:

Percent gastric survival=100*cfu_{Gast}/cfu₀

Germination was calculated indirectly by:

Percent germination 4 h=100*(cfu_{Gast}−cfu_{Bile})/cfu_{Gast}

Compared to the benchmark strains (Bacillus subtilis and Bacillus licheniformis strains) we have identified internal Bacillus strains that germinate fast.

TABLE 3
Gastric survival and germination

| | | | Gastric | Germination |
|---|---|---|---|---|
| Strain ID | Genus | Species | survival | 4 h |
| NRRL B-50136 | Bacillus | subtilis | 68% | 58% |
| NRRL B-50147 | Bacillus | amyloliquefaciens | 104% | 94% |
| NRRL B-50141 | Bacillus | amyloliquefaciens | 92% | 86% |
| ATCC PTA-7543 | Bacillus | amyloliquefaciens | 100% | 98% |
| NRRL B-50888 | Bacillus | amyloliquefaciens | 79% | 93% |
| ATCC PTA-7549 | Bacillus | amyloliquefaciens | 100% | 87% |
| NRRL B-50349 | Bacillus | amyloliquefaciens | 63% | 95% |
| Bacillus subtilis | Bacillus | subtilis | 74% | 40% |
| (isolated from | | | | |
| Kemin product, | | | | |
| clostat) | | | | |
| DSM 17229, Chr | Bacillus | subtilis | 73% | 45% |
| Hansen | | | | |
| Gallipro Tect, | Bacillus | licheniformis | 121% | 15% |
| isolated from Chr | | | | |
| Hansen product | | | | |

### AZCL Substrate Testing of Enzyme Activities

Commercially available AZCL substrates were incorporated into agar plates to monitor enzyme activity based on liberating a soluble blue dye fragment. Culture supernatants from an overnight culture were spotted on AZCL substrate plates and incubated for 30° C. for 24 h (Aerobic incubation) and 48 h (Anaerobic incubation). Observation of the liberated soluble blue dye as a result of enzyme hydrolysis served as evidence of enzyme activity present for a particular substrate. All samples and controls were run in duplicate wells. Non-inoculated wells were included as negative controls (n=6 wells), and enzyme controls (n=2 wells) were included as positive controls. The results are shown in Table 4 and Table 5 herein below.

Furthermore the results from Table 4 and Table 5, show strain to strain variation in enzyme activity both aerobically and anaerobically, with consistent positive activity on casein substrate under aerobic growth conditions.

TABLE 4
AZCL Activity - Aerobic

| NZ Strain ID | Amylose | Arabinan | Arabinoxylan | Casein | Cellulose | Xylan |
|---|---|---|---|---|---|---|
| NRRL B-50136 | + | + | + | + | − | + |
| NRRL B-50147 | + | + | + | + | − | + |
| NRRL B-50141 | + | + | + | + | + | + |
| PTA-7543 | + | + | + | + | − | + |
| NRRL B-50888 | + | + | + | + | + | + |
| PTA-7549 | + | + | + | + | − | + |
| NRRL B-50349 | + | + | + | + | + | + |

TABLE 5
ZCL Activity - Anaerobic

| NZ Strain ID | Amylose | Arabinan | Arabinoxylan | Casein | Cellulose | Xylan |
|---|---|---|---|---|---|---|
| NRRL B-50136 | + | + | + | + | − | + |
| NRRL B-50147 | + | + | + | + | − | + |
| NRRL B-50141 | + | + | + | + | + | + |
| PTA-7543 | + | + | + | + | − | + |
| NRRL B-50888 | + | + | + | + | + | + |
| PTA-7549 | + | + | + | + | − | + |
| NRRL B-50349 | + | − | + | + | − | + |

### Deposit of Biological Material

The following biological materials were deposited under the terms of the Budapest Treaty at the American Type Culture Collection (ATCC), 10801 University Blvd., Manassas, Va. 20108, USA, and the Microbial Genomics and Bioprocessing Research Unit (NRRL) National Center for Agricultural Utilization Research 1815 N. University Street, Peoria, Ill. 61604, USA and given the following accession numbers:

TABLE 6
Deposit of Biological Material

| Identification | Accession Number | Date of Deposit |
|---|---|---|
| Bacillus subtilis | NRRL B-50136 | 30-May-2010 |
| Bacillus amyloliquefaciens | NRRL B-50147 | 18-June-2008 |
| Bacillus amyloliquefaciens | NRRL B-50141 | 03-Jun-2008 |
| Bacillus amyloliquefaciens | PTA-7543 | 20-Apr-2006 |
| Bacillus amyloliquefaciens | NRRL B-50888 | 12-Dec-2013 |
| Bacillus amyloliquefaciens | PTA-7549 | 20-Apr-2006 |
| Bacillus amyloliquefaciens | NRRL B-50349 | 04-Mar-2010 |

NRRL B-50888 was isolated from a tire disposal site, Roanoke, Va., USA.

The strains have been deposited under conditions that assure that access to the culture will be available during the pendency of this patent application to one determined by foreign patent laws to be entitled thereto. The deposits represent a substantially pure culture of the deposited strain. The deposits are available as required by foreign patent laws in countries wherein counterparts of the subject application or its progeny are filed. However, it should be understood that the availability of a deposit does not constitute a license to practice the subject invention in derogation of patent rights granted by governmental action.

### Example 2

### Mucin Adhesion Assay

Bacillus vegetative cells were evaluated for the ability to adhere to mucin in vitro using the following assay.

Porcine stomach type II mucin was suspended in sterile water to 10 mg/ml before being added to a sterile plastic 96-well plate. The mucin was immobilized to individual wells of a plastic 96-well plate during a 1 hour incubation at 37° C. before overnight incubation at 4° C. The following day, unbound mucin was removed by gentle washing using sterile phosphate buffer. Then sterile bovine serum albumin (BSA) at 20 g/liter was added, followed by incubation for 2 hours at 4° C. Unbound BSA was then removed by 4-5 gentle washes with sterile phosphate buffer before approximately 1E8 CFU of Bacillus vegetative cell culture was added to the well. Bacillus cells were allowed to attach during a 1-hour incubation at 37° C. Each well was then washed gently five times with sterile phosphate buffer to remove unattached Bacillus cells. Those cells still attached to mucin are removed by adding sterile Triton X-100 at 0.5% (v/v) in water, with thorough mixing. Cells (100 ul) were then plated onto standard methods agar and incubated at 37° C. overnight before examination. Extensive Bacillus growth on the plates indicates a strain is positive for the ability to adhere to mucin. Little to no growth (below 10 colonies) indicates a strain is negative for mucin adherent ability. Candidate strains were tested in triplicate, and scored +/− for mucin adhesion ability. The results are summarized in Table 7.

### Biofilm Formation Assay:

Bacillus candidate strains were evaluated for the ability to form a biofilm in vitro using the following assay

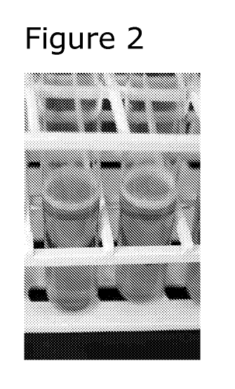

Approximately 1E8 of vegetative Bacillus cells from an overnight culture were inoculated into sterile tryptic soy broth at pH 7. Cells were incubated at 35° C. in static conditions (no shaking). After 24 and 48 hours of incubation, tubes were examined for a pellicle at the liquid:air interface. The formation of a pellicle indicates biofilm formation and results are recorded as +/− at each time point. Biofilm formation was shown as an example as the white pellicle at the liquid:air interface in strains ATCC PTA-7549 (left) and NRRL B-50349 (right) in FIG. 2. The results are summarized in Table 7.

Anti-Escherichia coli (CP) Assay:

Bacillus candidate strains were evaluated for the ability to inhibit the growth of Escherichia coli in vitro when cultured together in agar.

Overnight culture of Bacillus candidate strains to be tested and E. coli were grown at 35° C. in sterile tryptic soy broth with 0.6% yeast extract. E. coli culture (100 ul) was inoculated into 250 ml sterile tryptic soy agar with yeast extract (0.6%) with 1% agar after agar has cooled to about 40° C., but before it began to solidify. Bacillus overnight cultures were added (1 ul each) to the agar using a sterilized metal pin replicator. Both Bacillus and E. coli were co-incubated at 30° C. overnight in aerobic conditions, and then scored for Bacillus activity against E. coli, as determined by visual evidence of clearing zones. These zones indicate the Bacillus is able to inhibit the growth of E. coli culture embedded in the agar around it. Each strain was scored +/− for activity against individual E. coli isolates, which were tested separately.

Bacillus strains Clostat (Kemin), GallipoTect (Chr. Hansen), and DSM 17229 (Chr. Hansen) were tested against the following 4 E. coli isolates: 2008-10-1848-1, 73-70205-8, 74-61741-8, ATCC 10536. All other Bacillus strains examined were tested for activity against the following 8 E. coli isolates: DSA443, 73-70205-8, 2008-70-67-9, 74-61741-8, ATCC10536, ATCC25922, DSA449, 2008-10-1848-1. The results are summarized in Table 7.

TABLE 7
Biofilm formation, mucin adhesion and anti-E. coli activity

| | | | Biofilm | Biofilm | | |
|---|---|---|---|---|---|---|
| | | | Formation | Formation | Mucin | |
| Strain ID | Genus | Species | 24 h | 48 h | Adhesion | Anti-E. coli |
| NRRL B-50136 | Bacillus | subtilis | + | + | + | 0/8 |
| NRRL B-50147 | Bacillus | amyloliquefaciens | + | + | + | 0/8 |
| NRRL B-50141 | Bacillus | amyloliquefaciens | + | + | + | 2/8 |
| ATCC PTA-7543 | Bacillus | amyloliquefaciens | + | + | + | 7/8 |
| NRRL B-50888 | Bacillus | amyloliquefaciens | + | + | + | 7/8 |
| ATCC PTA-7549 | Bacillus | amyloliquefaciens | + | + | + | 8/8 |
| NRRL B-50349 | Bacillus | amyloliquefaciens | + | + | + | 6/8 |
| Bacillus subtilis | Bacillus | subtilis | + | + | + | 4/4 |
| (isolated from | | | | | | |
| Kemin product, | | | | | | |
| clostat) | | | | | | |
| DSM 17229, Chr | Bacillus | subtilis | − | − | + | 0/4 |
| Hansen | | | | | | |
| Gallipro Tect, | Bacillus | licheniformis | − | + | + | 0/4 |
| isolated from Chr | | | | | | |
| Hansen product | | | | | | |

1-15. (canceled)

16. A method for improving the performance of poultry or swine comprising feeding the poultry or swine with a Bacillus subtilis strain or a Bacillus amyloliquefaciens strain characterized in that
(a) at least 60% of the Bacillus spores survive gastric stability;
(b) at least 55% of the Bacillus spores germinate in less than 4 hours,
(c) the Bacillus spores have antimicrobial activity against Clostridium perfringens and
(d) the Bacillus strain has enzyme activity under aerobic conditions against one or more of the substrates selected from the group consisting of amylose, arabinan, arabinoxylan, casein and xylan.

17. The method of claim 16, wherein the Bacillus strain is selected from the group consisting of
the strain having the deposit accession number NRRL B-50136 or a strain having all of the identifying characteristics of Bacillus NRRL B-50136 or a mutant thereof;
the strain having the deposit accession number NRRL B-50147 or a strain having all of the identifying characteristics of Bacillus NRRL B-50147 or a mutant thereof;
the strain having the deposit accession number NRRL B-50141 or a strain having all of the identifying characteristics of Bacillus NRRL B-50141 or a mutant thereof;
the strain having the deposit accession number PTA-7543 or a strain having all of the identifying characteristics of Bacillus PTA-7543 or a mutant thereof;
the strain having the deposit accession number NRRL B-50888 or a strain having all of the identifying characteristics of Bacillus NRRL B-50888 or a mutant thereof;
the strain having the deposit accession number PTA-7549 or a strain having all of the identifying characteristics of Bacillus PTA-7549 or a mutant thereof;
the strain having the deposit accession number B-50349 or a strain having all of the identifying characteristics of Bacillus B-50349 or a mutant thereof; or any combination thereof.

18. A method for improving the performance of poultry or swine comprising feeding the poultry or swine with a Bacillus strain selected from the group consisting of
the strain having the deposit accession number NRRL B-50136 or a strain having all of the identifying characteristics of Bacillus NRRL B-50136 or a mutant thereof;
the strain having the deposit accession number NRRL B-50147 or a strain having all of the identifying characteristics of Bacillus NRRL B-50147 or a mutant thereof;
the strain having the deposit accession number NRRL B-50141 or a strain having all of the identifying characteristics of Bacillus NRRL B-50141 or a mutant thereof;
the strain having the deposit accession number PTA-7543 or a strain having all of the identifying characteristics of Bacillus PTA-7543 or a mutant thereof;
the strain having the deposit accession number NRRL B-50888 or a strain having all of the identifying characteristics of Bacillus NRRL B-50888 or a mutant thereof;
the strain having the deposit accession number PTA-7549 or a strain having all of the identifying characteristics of Bacillus PTA-7549 or a mutant thereof;
the strain having the deposit accession number B-50349 or a strain having all of the identifying characteristics of Bacillus B-50349 or a mutant thereof; or any combination thereof.

19. The method of claim 16, wherein the improved performance comprises one or more improvements selected from the group consisting of increased body weight gain, improved feed conversion rate, improving the feed efficiency, improving health and decreased mortality.

20. The method of claim 16, wherein the method comprises administration of a bacterial count of each Bacillus (e.g. in spore form) between 1×10⁵ and 1×10¹⁵ CFU/animal/day, preferably between 1×10⁷ and 1×10¹³ CFU/animal/day, and more preferably between 1×10⁸ and 1×10¹² CFU/animal/day.

21. A poultry or swine feed comprising a Bacillus subtilis strain or a Bacillus amyloliquefaciens strain characterized in that
(a) at least 60% of the Bacillus spores survive gastric stability;
(b) at least 55% of the Bacillus spores germinate in less than 4 hours,
(c) the Bacillus spores have antimicrobial activity against Clostridium perfringens and
(d) the Bacillus strain has enzyme activity under aerobic conditions against one or more of the substrates selected from the group consisting of amylose, arabinan, arabinoxylan, casein and xylan.
